(19) United States
(12) Patent Application Publication
(10) Pub. No.: US 2023/0149521 A1
(43) Pub. Date: May 18, 2023

(54) METHODS OF TREATING VIRAL INFECTIONS USING ARGINASE

(71) Applicant: Bio-Cancer Treatment International Limited, Shatin, Hong Kong (CN)

(72) Inventors: Kwok-Ming Cheung, Shatin, Hong Kong (CN); Paul Cheng, Shatin, Hong Kong (CN)

(21) Appl. No.: 17/919,223

(22) PCT Filed: Apr. 16, 2021
(86) PCT No.: PCT/CN2021/087713

§ 371 Date: Oct. 14, 2022

Related U.S. Application Data
(60) Provisional application No. 63/011,658, filed on Apr. 17, 2020.

(51) Int. Cl. A61K 38/50; A61K 47/60; A61P 31/14; A61P 31/16; A61P 31/20; A61K 45/06

(52) U.S. Cl. CPC A61K 38/50; A61K 47/60; A61P 31/14; A61P 31/16; A61P 31/20; A61K 45/06; C12Y 305/03001

(57) ABSTRACT

Provided are use of an arginase (e.g., a non-PEGylated arginase or a PEGylated arginase) in the treatment of viral-associated diseases or disorders, use of arginase in the treatment of other pathogen-associated diseases or disorders, and kits comprising arginase for said uses.

### RELATED APPLICATIONS

This application claims priority to and the benefit of U.S. Provisional Patent Application No. 63/011,658, filed Apr. 17, 2020, the entire disclosure of which is incorporated herein by reference for all purposes.

### FIELD OF THE INVENTION

The invention relates to use of an arginase (e.g., a PEGylated arginase) in the treatment of virus-associated diseases or disorders, use of an arginase (e.g., a PEGylated arginase) in the treatment of other pathogen-associated diseases or disorders, and kits comprising arginase (e.g., a PEGylated arginase) for said uses.

### BACKGROUND

Despite advances made in the treatment of infectious diseases and disorders, there continue to be treatment challenges. For example, a pathogen for treatment may develop resistance to a treatment agent. Development of therapeutic resistance frequently occurs in the context of antibiotic-resistant bacteria (Blair et al., Molecular mechanisms of antibiotic resistance. Nat Rev Microbiol. 2015 January; 13(1):42-51.)

Additionally, no treatment may be available for inhibiting a pathogen of interest. Lack of a suitable treatment agent is encountered frequently in the context of treating many types of viruses. Treatment of viral diseases and disorders remains particularly challenging, due to the lack of available antiviral therapies.

Infectious diseases, such as those caused by viral infection, continue to present a significant public health risk. According to the U.S. Center for Disease Control and Prevention, the Ebolavirus outbreak of 2014 resulted in approximately 29,000 new cases, of which over 11,000 resulted in death. According to the World Health Organization, as of 30 Mar. 2020, the coronavirus pandemic of 2019 to 2020 caused by the virus SARS-CoV-2 (COVID-19), resulted in more than 785,000 cases and almost 38,000 deaths. Beyond the risk of infection from potentially lethal viral disease, many individuals live with chronic, recurrent viral infection, such as human papillomavirus (HPV) or herpes simplex virus (HSV).

Accordingly, there remains a critical need for new and effective treatments for infectious diseases and disorders, including diseases and disorders associated with viruses. There is also a need for broad spectrum antiviral therapeutics that are effective against multiple virus species and strains.

### SUMMARY

Described herein is a method of using an arginase (e.g., a non-PEGylated or a PEGylated arginase) in the treatment of a disease or disorder, for example, a virus-associated disease or disorder. For example, described herein is a method of treating a disease or disorder, for example, a virus-associated disease or disorder, where the method includes administering a therapeutically effective amount of a composition comprising an arginase (e.g., a non-PEGylated or a PEGylated arginase), or a pharmaceutically acceptable salt thereof. In some embodiments the method includes administering a therapeutically effective amount of the composition to a patient, for example, a patient in need of treatment.

In some embodiments, the arginase (e.g., a non-PEGylated or a PEGylated arginase), or a pharmaceutically acceptable salt thereof, or a composition comprising the arginase, is effective to inhibit genomic replication of a virus. In some embodiments, the arginase, or a composition comprising the arginase, or a pharmaceutically acceptable salt thereof, is effective to inhibit transmission of a virus. In some embodiments, the arginase, or a pharmaceutically acceptable salt thereof, or a composition comprising the arginase, is effective to inhibit viral gene expression. In some embodiments, the arginase, or a pharmaceutically acceptable salt thereof, or a composition comprising the arginase, is effective to inhibit assembly of a virus. Kits containing an arginase, or a pharmaceutically acceptable salt thereof, or a composition comprising the arginase, or a pharmaceutically acceptable salt thereof, are also described herein.

The amino acid arginine, while critical for processes in mammalian cells such as cell proliferation and growth, is considered to be a non-essential amino acid in humans due to the ability to metabolize citrulline to derive an arginine pool (Hecker et al. (1990) Proc. Natl. Acad. Sci. USA. 87(21): 8612-6). In contrast, many viruses are reliant on exogenous arginine for protein synthesis (U.S. Pat. No. 9,011,845 B2). The inventors have discovered that arginase and PEGylated-arginase are effective to deplete arginine levels and presents an unexpected mechanism for treatment of viral infection, with the ability to target a broad spectrum of viruses. Additionally, the inventors have discovered that arginine depletion by non-PEGylated arginase or PEGylated arginase provides a method of treating previously unencountered viruses, or viruses for which no approved therapeutic currently exists, such as SARS-CoV-2 (COVID-19).

Described herein is a method of treating a disease or disorder, for example, virus-associated disease or disorder, where the method includes administering to a patient in need thereof a therapeutically effective amount of a composition comprising an arginase (e.g., a non-PEGylated or a PEGylated arginase), or a pharmaceutically acceptable salt thereof. In some embodiments described herein, the PEGylated arginase comprises at least one polyethylene glycol (PEG) molecule conjugated to an arginase sequence.

In some embodiments, the arginase has at least about 90% sequence identity to a protein sequence of SEQ ID NO:1-41, or a fragment thereof. In some embodiments, the arginase has at least about 90% sequence identity to a protein sequence selected from the group consisting of SEQ ID NO:3-41, or a fragment thereof. In some embodiments, the arginase has at least about 90% sequence identity to a protein sequence of SEQ ID NO:1 or 2, or a fragment thereof. In some embodiments, the arginase comprises a protein sequence selected from the group consisting of SEQ ID NO:1-41. In some embodiments, the arginase comprises a protein sequence selected from the group consisting of SEQ ID NO:3-41. In some embodiments, the arginase comprises a protein sequence of SEQ ID NO:1 or 2.

Thus, in some embodiments described herein, the method includes administering to a patient in need thereof a therapeutically effective amount of a composition comprising an arginase, or a pharmaceutically acceptable salt thereof, wherein the arginase: has at least about 90% sequence identity to a protein sequence of SEQ ID NO:1-41, or a fragment thereof; has at least about 90% sequence identity to a protein sequence selected from the group consisting of SEQ ID NO:3-41, or a fragment thereof; has at least about 90% sequence identity to a protein sequence of SEQ ID NO:1 or 2, or a fragment thereof; comprises a protein sequence selected from the group consisting of SEQ ID NO:1-41; comprises a protein sequence selected from the group consisting of SEQ ID NO:3-41; or comprises a protein sequence of SEQ ID NO:1 or 2.

Accordingly, in one aspect, the present disclosure provides a method of treating a virus-associated disease or disorder, the method comprising administering to a patient in need thereof a therapeutically effective amount of a composition comprising an arginase, or a pharmaceutically acceptable salt thereof, wherein the non-PEGylated or the PEGylated arginase has at least about 90% sequence identity to SEQ ID NO:1 or 2, or a fragment thereof, and wherein the virus-associated disease or disorder is associated with a virus selected from the group consisting of a coronavirus, a papillomavirus, a pneumovirus, a picornavirus, a flavivirus, an alphavirus, an ebolavirus, a morbillivirus, an enterovirus, an orthopneumovirus, a lentivirus, and a hepatovirus. In some embodiments, the arginase is a PEGylated-arginase comprising at least one polyethylene glycol molecule conjugated to the arginase. In some embodiments, the virus-associated disease or disorder is a virus infection. For example, in some embodiments, the virus-associated disease or disorder is a virus infection selected from the group consisting of: a coronavirus infection, a papillomavirus infection, a pneumovirus infection, a picornavirus infection, a flavivirus infection, an alphavirus infection, an ebolavirus infection, a morbillivirus infection, an enterovirus infection, an orthopneumovirus infection, a lentivirus infection, and a hepatovirus infection.

In another aspect, the present disclosure provides a method of treating a virus-associated disease or disorder, the method comprising administering to a patient in need thereof a therapeutically effective amount of a composition comprising an arginase, or a pharmaceutically acceptable salt thereof, wherein the non-PEGylated or the PEGylated arginase has at least about 90% sequence identity to a protein sequence selected from the group consisting of SEQ ID NO:3-50 and 56, or a fragment thereof. In some embodiments, the arginase is a PEGylated-arginase comprising at least one polyethylene glycol molecule conjugated to the arginase. In some embodiments, the virus-associated disease or disorder is associated with a virus selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebolavirus, a morbillivirus, an enterovirus, an orthopneumovirus, a lentivirus, and a hepatovirus. In some embodiments, the virus-associated disease or disorder is a virus infection. For example, in some embodiments, the virus-associated disease or disorder is a virus infection selected from the group consisting of: an RNA virus infection, a DNA virus infection, a coronavirus infection, a papillomavirus infection, a pneumovirus infection, a picornavirus infection, an influenza virus infection, an adenovirus infection, a cytomegalovirus infection, a polyomavirus infection, a poxvirus infection, a flavivirus infection, an alphavirus infection, an ebolavirus infection, a morbillivirus infection, an enterovirus infection, an orthopneumovirus infection, a lentivirus infection, and a hepatovirus infection.

In some embodiments, the virus-associated disease or disorder is localized to or affects an organ or a tissue of the patient. In some embodiments, the virus-associated disease or disorder comprises a virus infection of an organ or a tissue of the patient. In certain embodiments, the organ or the tissue can be, but is not limited to, an eye, an ear, an inner ear, a lung, a trachea, a bronchus, bronchioli, a liver, a gall bladder, a bile duct, a kidney, a bladder, a testicle, a cervix, an ovary, a uterus, skin, or a brain. For example, in certain embodiments, the organ or the tissue is selected from the group consisting of an eye, an ear, an inner ear, a lung, a trachea, a bronchus, bronchioli, a liver, a gall bladder, a bile duct, a kidney, a bladder, a testicle, a cervix, an ovary, a uterus, skin, and a brain.

In some embodiments, the virus-associated disease or disorder can be, but is not limited to: acute respiratory distress syndrome; chronic obstructive pulmonary disease (COPD); pneumonia; drug-resistant pneumonia; hand, foot, and mouth disease; atopic asthma; or non-atopic asthma. In some embodiments, the virus-associated disease or disorder is selected from the group consisting of: acute respiratory distress syndrome; COPD; pneumonia; drug-resistant pneumonia; hand, foot, and mouth disease; atopic asthma; and non-atopic asthma.

In various embodiments, disclosed herein is a method of inhibiting genomic replication of a virus, a method of inhibiting transmission of a virus, a method of inhibiting assembly of a virus, a method of inhibiting virus gene expression, and a method of inhibiting virus release. The aforementioned methods can include administering to a patient in need thereof a therapeutically effective amount of a composition comprising an arginase (for example, an arginase comprising an amino acid sequence of SEQ ID NO:1 or 2 or an arginase comprising an amino acid sequence selected from the group consisting of SEQ ID NO:3-50 and 56), or a pharmaceutically acceptable salt thereof. For example, provided herein is a method of inhibiting genomic replication of a virus, the method comprising administering to a patient in need thereof a therapeutically effective amount of a composition comprising an arginase comprising an amino acid sequence of SEQ ID NO:1 or 2, or a pharmaceutically acceptable salt thereof, wherein the arginase has at least about 90% sequence identity to SEQ ID NO:1 or 2, or a fragment thereof, and wherein the virus is selected from the group consisting of a coronavirus, a papillomavirus, a pneumovirus, a picornavirus, a flavivirus, an alphavirus, an ebolavirus, a morbillivirus, an enterovirus, an orthopneumovirus, a lentivirus, and a hepatovirus. In some embodiments, the arginase is a PEGylated-arginase comprising at least one polyethylene glycol molecule conjugated to the arginase.

In another aspect, provided herein is a method of inhibiting genomic replication of a virus, the method comprising administering to a patient in need thereof a therapeutically effective amount of a composition comprising an arginase, or a pharmaceutically acceptable salt thereof, wherein the arginase has at least about 90% sequence identity to a protein sequence selected from the group consisting of SEQ ID NO:3-50 and 56, or a fragment thereof. In some embodiments, the arginase is a PEGylated-arginase comprising at least one polyethylene glycol molecule conjugated to the arginase. In some embodiments, the virus is selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebolavirus, a morbillivirus, an enterovirus, an orthopneumovirus, a lentivirus, and a hepatovirus.

In another aspect, provided herein is a method of inhibiting transmission of a virus, the method comprising administering to a patient in need thereof a therapeutically effective amount of a composition comprising an arginase, or a pharmaceutically acceptable salt thereof, and wherein the arginase has at least about 90% sequence identity to a protein sequence comprising an arginase amino acid sequence of SEQ ID NO:1 or 2, wherein the virus is selected from the group consisting of a coronavirus, a papillomavirus, a pneumovirus, a picornavirus, a flavivirus, an alphavirus, an ebolavirus, a morbillivirus, an enterovirus, an orthopneumovirus, a lentivirus, and a hepatovirus. In some embodiments, the arginase is a PEGylated-arginase comprising at least one polyethylene glycol molecule conjugated to the arginase.

In another aspect, provided herein is a method of inhibiting transmission of a virus, the method comprising administering to a patient in need thereof a therapeutically effective amount of a composition comprising an arginase, or a pharmaceutically acceptable salt thereof, and wherein the arginase has at least about 90% sequence identity to a protein sequence selected from the group consisting of SEQ ID NO:3-50 and 56, or a fragment thereof. In some embodiments, the arginase is a PEGylated-arginase comprising at least one polyethylene glycol molecule conjugated to the arginase. In some embodiments, the virus is selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebolavirus, a morbillivirus, an enterovirus, an orthopneumovirus, a lentivirus, and a hepatovirus.

In another aspect, provided herein is a method of inhibiting assembly of a virus, the method comprising administering to a patient in need thereof a therapeutically effective amount of a composition comprising an arginase, or a pharmaceutically acceptable salt thereof, wherein the arginase has at least about 90% sequence identity to a protein sequence selected from the group consisting of SEQ ID NO:1-50 and 56, or a fragment thereof. In some embodiments, the arginase is a PEGylated-arginase comprising at least one polyethylene glycol molecule conjugated to the arginase.

In another aspect, provided herein is a method of inhibiting virus gene expression, the method comprising administering to a patient in need thereof a therapeutically effective amount of a composition comprising an arginase, or a pharmaceutically acceptable salt thereof, wherein the arginase has at least about 90% sequence identity to a protein sequence selected from the group consisting of SEQ ID NO:1-50 and 56, or a fragment thereof. In some embodiments, the arginase is a PEGylated-arginase comprising at least one polyethylene glycol molecule conjugated to the arginase. In some embodiments, the inhibiting comprises inhibiting gene expression of a specific group of genes. For example, in some embodiments, the inhibiting comprises inhibiting β gene expression, γ gene expression, or 13 gene expression and γ gene expression.

In another aspect, provided herein is a method of inhibiting virus release, the method comprising administering to a patient in need thereof a therapeutically effective amount of a composition comprising an arginase, or a pharmaceutically acceptable salt thereof, wherein the arginase has at least about 90% sequence identity to a protein sequence selected from the group consisting of SEQ ID NO:1-50 and 56, or a fragment thereof. In some embodiments, the arginase is a PEGylated-arginase comprising at least one polyethylene glycol molecule conjugated to the arginase. In some embodiments, the virus is selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebolavirus, a morbillivirus, an enterovirus, an orthopneumovirus, a lentivirus, and a hepatovirus.

In some embodiments described herein, the method of treating a virus-associated disease or disorder includes a method of treating a virus-associated disease or disorder associated with a coronavirus. Also described herein are a method of inhibiting genomic replication of a virus, a method of inhibiting transmission of a virus, a method of inhibiting assembly of a virus, a method of inhibiting virus gene expression, and a method of inhibiting virus release, wherein the virus is a coronavirus. In certain embodiments, the coronavirus is selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2 (COVID-19).

In some embodiments described herein, the method of treating a virus-associated disease or disorder includes a method of treating a virus-associated disease or disorder associated with an influenza virus. Also described herein are a method of inhibiting genomic replication of a virus, a method of inhibiting transmission of a virus, a method of inhibiting assembly of a virus, a method of inhibiting virus gene expression, and a method of inhibiting virus release, wherein the virus is an influenza virus. In certain embodiments, the influenza virus is selected from the group consisting of influenza virus A (e.g., H1N1 and H5N1), influenza virus B, influenza virus C, influenza virus D.

In some embodiments described herein, the method of treating a virus-associated disease or disorder includes a method of treating a virus-associated disease or disorder associated with an adenovirus. Also described herein are a method of inhibiting genomic replication of a virus, a method of inhibiting transmission of a virus, a method of inhibiting assembly of a virus, a method of inhibiting virus gene expression, and a method of inhibiting virus release, wherein the virus is an adenovirus. In certain embodiments, the adenovirus is AdV5.

In some embodiments described herein, a virus is a drug-resistant virus.

In some embodiments, a method described herein further comprises administering a composition comprising an antiviral agent. In some embodiments the administering includes administering a therapeutically effective amount of a composition comprising an antiviral agent. In some embodiments the administering a composition comprising an antiviral agent is to a patient, for example, a patient in need of treatment. In some embodiments, the antiviral agent is selected from the group consisting of lamivudine, an interferon alpha composition (e.g., Interferon alfa (INN; HuIFN-alpha-Le)), a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), Rifampicin, zanamivir, peramivir, danoprevir, ritonavir, remdesivir, and oseltamivir. In certain embodiments, the virus to be treated is an influenza virus, and the composition comprises zanamivir. In certain embodiments, the virus to be treated is a hepatitis virus, e.g., hepatitis B virus, and the composition comprises lamivudine. In certain embodiments, the administering of the antiviral agent is before, during, or after the administering of the arginase. For example, described herein is a method of treating a disease or disorder, for example, virus-associated disease or disorder, where the method includes administering to a patient in need thereof a therapeutically effective amount of a composition comprising an arginase (e.g., a PEGylated arginase), or a pharmaceutically acceptable salt thereof, and the method further includes administering (for example, administering to a patient in need thereof) a composition comprising an antiviral agent (for example a therapeutically effective amount of a composition comprising an antiviral agent).

Also described herein are a method of inhibiting genomic replication of a virus, a method of inhibiting transmission of a virus, a method of inhibiting assembly of a virus, a method of inhibiting virus gene expression, and a method of inhibiting virus release, wherein the described method includes administering to a patient (for example, a patient in need thereof) a therapeutically effective amount of a composition comprising an arginase, or a pharmaceutically acceptable salt thereof, and the method further includes administering (for example, administering to a patient in need thereof) a composition comprising an antiviral agent (for example a therapeutically effective amount of a composition comprising an antiviral agent).

In another aspect, provided herein is a method of treating a bacterial disease or disorder, the method comprising administering to a patient in need thereof a therapeutically effective amount of a composition comprising an arginase selected from the group consisting of SEQ ID NO:1 or 2, or a pharmaceutically acceptable salt thereof, wherein the arginase has at least about 90% sequence identity to SEQ ID NO:1 or 2, or a fragment thereof, and wherein the bacterial disease or disorder is associated with a bacteria selected from the group consisting of: Streptococcus pneumoniae, Mycoplasma pneumoniae, Haemophilus influenzae, Legionella pneumophila, Salmonella enterica, Salmonella bongori, Escherichia coli, Helicobacter pylori, Neisseria gonorrhoeae, Neisseria meningitidis, Staphylococcus aureus, Acinetobacter baumannii, Burkholderia cepacian, Clostridium difficile, Clostridium sordellii, an Enterobacteriaceae, Enterococcus faecalis, Klebsiella pneumoniae, Morganella morganii, Mycobacterium abscessus, Mycobacterium tuberculosis, a Norovirus, Pseudomonas aeruginosa, and Stenotrophomonas maltophilia. In some embodiments, the arginase is a PEGylated-arginase comprising at least one polyethylene glycol molecule conjugated to the arginase.

In another aspect, provided herein is a method of treating a bacterial disease or disorder, the method comprising administering to a patient in need thereof a therapeutically effective amount of a composition comprising an arginase, or a pharmaceutically acceptable salt thereof, wherein the arginase has at least about 90% sequence identity to a protein sequence selected from the group consisting of SEQ ID NO:3-50 and 56, or a fragment thereof. In some embodiments, the arginase is a PEGylated-arginase comprising at least one polyethylene glycol molecule conjugated to the arginase. In some embodiments, the bacterial disease or disorder is associated with a bacteria selected from the group consisting of: Chlamydia pneumoniae, Vibrio cholerae, Streptococcus pneumoniae, Mycoplasma pneumoniae, Haemophilus influenzae, Legionella pneumophila, Salmonella enterica, Salmonella bongori, Escherichia coli, Helicobacter pylori, Neisseria gonorrhoeae, Neisseria meningitidis, Staphylococcus aureus, Acinetobacter baumannii, Burkholderia cepacian, Clostridium difficile, Clostridium sordellii, an Enterobacteriaceae, Enterococcus faecalis, Klebsiella pneumoniae, Morganella morganii, Mycobacterium abscessus, Mycobacterium tuberculosis, a Norovirus, Psuedomonas aeruginosa, and Stenotrophomonas maltophilia.

In another aspect, provided herein is a method of treating a fungal disease or disorder, the method comprising administering to a patient in need thereof a therapeutically effective amount of a composition comprising an arginase, or a pharmaceutically acceptable salt thereof, wherein the arginase has at least about 90% sequence identity to a protein sequence selected from the group consisting of SEQ ID NO:1-50 and 56, or a fragment thereof. In some embodiments, the arginase is a PEGylated-arginase comprising at least one polyethylene glycol molecule conjugated to the arginase. In some embodiments, the fungal disease is associated with a fungus selected from the group consisting of a Pneumocystis fungus, an Aspergillus fungus, and a Candida fungus.

In another aspect, provided herein is a method of treating an amoeba disease or disorder, the method comprising administering to a patient in need thereof a therapeutically effective amount of a composition comprising an arginase, or a pharmaceutically acceptable salt thereof, wherein the arginase has at least about 90% sequence identity to a protein sequence selected from the group consisting of SEQ ID NO:1-50 and 56, or a fragment thereof. In some embodiments, the arginase is a PEGylated-arginase comprising at least one polyethylene glycol molecule conjugated to the arginase. In some embodiments, the amoeba disease or disorder is associated with an amoeba selected from the group consisting of Dientamoeba fragilis, Entamoeba histolytica, Naegleria fowleri, an Acanthamoeba, Acanthamoeba keratitis, Balamuthia mandrillaris, and Sappinia diploidea.

In some embodiments, the arginase is administered at a dose of about 1 ng/kg body weight per day to about 1 mg/kg body weight per day.

In some embodiments, the administering is topical, parenteral, oral, pulmonary, intratracheal, intranasal, intrathecal, transdermal, subcutaneous, intraocular, intravitreal, intraperitoneal, or intraduodenal administration. A dose may include or consist essentially of about 1 ng/kg to 1 mg/kg of an arginase, or a pharmaceutically acceptable salt thereof, as described herein.

In some embodiments, the arginase comprises an amino acid sequence that includes a protein tag sequence. For example, in some embodiments, the arginase comprises the amino acid sequence of SEQ ID NO:1-43 and a protein tag sequence. In some embodiments, the arginase is a PEGylated-arginase comprising at least one polyethylene glycol molecule conjugated to the arginase. In certain embodiments, the protein tag sequence is a 6xHis tag sequence of SEQ ID NO:51. In certain embodiments, the protein tag sequence is located at the amino terminus of the arginase. In certain embodiments, the protein tag sequence is located at the carboxy terminus of the arginase.

In some embodiments, the PEGylated-arginase comprises 2, 3, 4, or more polyethylene glycol molecules conjugated to the arginase sequence. In some embodiments, the polyethylene glycol molecule is about 5 kDa, about 10 kDa, about 15 kDa, about 20 kDa, about 30 kDa, or about 40 kDa. In certain embodiments, the polyethylene glycol is from about 10 kDa to about 30 kDa or from about 20 kDa to about 40 kDa.

In some embodiments, the composition further comprises a non-native metal cofactor. In certain embodiments, the non-native metal cofactor is selected from the group consisting of cobalt, manganese, iron, and zinc.

In another aspect, the present disclosure provides a composition comprising:

an arginase, or a pharmaceutically acceptable salt thereof, wherein the arginase has at least about 90% sequence identity to a protein sequence selected from the group consisting of SEQ ID NO:1-50 and 56, or a fragment thereof;

an antiviral agent; and

a pharmaceutically acceptable excipient.

In some embodiments, the arginase is a PEGylated-arginase comprising at least one polyethylene glycol molecule conjugated to the arginase. In some embodiments, the antiviral agent included in a composition disclosed herein is selected from the group consisting of lamivudine, an interferon alpha composition (e.g., Interferon alfa (INN; HuIFN-alpha-Le)), a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), Rifampicin, zanamivir, peramivir, danoprevir, ritonavir, remdesivir, and oseltamivir.

In some embodiments, the arginase included in a composition disclosed herein includes a protein tag sequence. In some embodiments, the protein tag sequence is a 6xHis tag sequence of SEQ ID NO:51. In certain embodiments, the protein tag sequence is located at the amino terminus of the arginase. In certain embodiments, wherein the protein tag sequence is located at the carboxy terminus of the arginase.

In some embodiments, the PEGylated-arginase included in a composition disclosed herein comprises 2, 3, 4, or more polyethylene glycol molecules conjugated to the arginase sequence. In some embodiments, the polyethylene glycol molecule is about 5 kDa, about 10 kDa, about 15 kDa, about 20 kDa, about 30 kDa, or about 40 kDa. In certain embodiments, the polyethylene glycol molecule is from about 10 kDa to about 30 kDa or from about 20 kDa to about 40 kDa.

In some embodiments, a composition described herein further comprises a non-native metal cofactor. In certain embodiments, the non-native metal cofactor is selected from the group consisting of cobalt, manganese, iron, and zinc.

In another aspect, provided herein is a kit comprising:

an arginase, or a pharmaceutically acceptable salt thereof, wherein the arginase has at least about 90% sequence identity to a protein sequence selected from the group consisting of SEQ ID NO:1-50 and 56, or a fragment thereof; and buffers, reagents, and detailed instructions for inhibiting production of a virus.

In some embodiments, the arginase is a PEGylated-arginase comprising at least one polyethylene glycol molecule conjugated to the arginase. In some embodiments, the kit further comprises an antiviral agent. In certain embodiments, the antiviral agent is selected from the group consisting of lamivudine, an interferon alpha composition (e.g., Interferon alfa (INN; HuIFN-alpha-Le)), a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), Rifampicin, zanamivir, peramivir, danoprevir, ritonavir, remdesivir, and oseltamivir.

In some embodiments, the kit is for inhibiting production of a coronavirus. In certain embodiments, the coronavirus is selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2 (COVID-19).

In some embodiments, the arginase included in a kit described herein includes a protein tag sequence. In certain embodiments, the protein tag sequence is a 6xHis tag sequence of SEQ ID NO:51. In certain embodiments, the protein tag sequence is located at the amino terminus of the arginase. In certain embodiments, the protein tag sequence is located at the carboxy terminus of the arginase.

In some embodiments, the PEGylated-arginase included in a kit described herein comprises 2, 3, 4, or more polyethylene glycol molecules conjugated to the arginase sequence. In some embodiments, the polyethylene glycol is about 5 kDa, about 10 kDa, about 15 kDa, about 20 kDa, about 30 kDa, or about 40 kDa. In certain embodiments, the polyethylene glycol is from about 10 kDa to about 30 kDa or from about 20 kDa to about 40 kDa.

In some embodiments, a kit described herein further comprises a non-native metal cofactor. In certain embodiments, the non-native metal cofactor is selected from the group consisting of cobalt, manganese, iron, and zinc.

### BRIEF DESCRIPTION OF THE FIGURES

FIG. 1 depicts a graph showing the dose-dependent inhibitory effect of PEGylated arginase on the replication of SARS-CoV. “*” and “**” denote p<0.05 and 0.01, respectively, in Dunnett's tests.

FIG. 2 depicts a graph showing the dose-dependent inhibitory effect of PEGylated arginase on the replication of SARS-CoV-2. “*” denotes p<0.05 in Dunnett's tests.

FIG. 3 depicts a graph showing the inhibitory effect of non-PEGylated arginase and PEGylated arginase on the replication of MERS-CoV. “% of inhibition” was calculated as (1− C_{[Arginase]}/C₀)×100%, where C_{[Arginase]} and C₀ are the viral genome copy number in culture supernatant after treating with a specific concentration of non-PEGylated arginase or PEGylated arginase and without arginase, respectively. Each dot represents one independent measurement. Gray curve represents a best-fitted two-parameters logistic model.

FIG. 4 depicts a graph showing the inhibitory effect of non-PEGylated arginase and PEGylated arginase on the replication of H1N1 influenza A virus (pandemic H1N1/09). “% of inhibition” was calculated as (1−C_{[Arginase]}/C₀)×100%, where C_{[Arginase]} and C₀ are the viral genome copy numbers in culture supernatant after treating with a specific concentration of non-PEGylated arginase or PEGylated arginase and without arginase, respectively. Each dot represents one independent measurement. Gray curve represents a best-fitted two-parameters logistic model.

FIG. 5 depicts a graph showing the dose-dependent inhibitory effect of PEGylated arginase on the replication of H5N1 influenza A virus (A/Vietnam/2013/04). “***” denotes p<0.001 in Dunnett's tests.

FIG. 6 depicts a graph showing the inhibitory effect of non-PEGylated arginase and PEGylated arginase on the replication of Human adenovirus serotype 5. “% of inhibition” was calculated as (1−C_{[Arginase]}/C₀)×100%, where C_{[Arginase]} and C₀ are the viral genome copy numbers in culture supernatant after treating with a specific concentration of non-PEGylated arginase or PEGylated arginase and without arginase, respectively. Each dot represents one independent measurement. Gray curve represents a best-fitted two-parameters logistic model.

Various aspects and embodiments of the invention are described in further detail below.

### DETAILED DESCRIPTION

The present disclosure provides a method of using PEGylated-arginase in the treatment of diseases and disorders, for example, virus-associated diseases and disorders.

The terms “a” and “an” as used herein mean “one or more” and include the plural unless the context is inappropriate.

As used herein, the term “PEGylated-arginase” means an arginase protein sequence modified by conjugation of at least one molecule of polyethylene glycol (PEG) and the term “non-PEGylated arginase” means an arginase protein sequence not having a molecule of PEG conjugated thereto. Without being bound by theory, it is believed that conjugation of PEG to a protein, for example, arginase increases protein stability, increases protein circulatory time, and minimizes immunoreactivity. “PEGylation,” as used herein, refers to the process of conjugating one or more molecules of PEG to an amino acid, for example, an amino acid of a protein or peptide, for example, an arginase protein. PEGylation can be achieved through covalent or non-covalent attachment of PEG to a compound of interest. Preferably, PEGylation is achieved through covalent PEG attachment. PEGylation can be achieved, for example, through conjugation of PEG to an amine group, thiol conjugation, oxidation of carbohydrates, N-terminal amino acid conjugation, or transglutaminase mediated enzymatic conjugation. Methods of PEGylation are known in the art and are described, for example, in Fee and Babu (2010) “Protein PEGylation: An overview of chemistry and process considerations,” European Pharmaceutical Review, 1:1-24. Proteins, peptides, and amino acids that have undergone PEGylation are referred to herein as “PEGylated” proteins, peptides, and amino acids.

In some embodiments, the arginase is a human arginase, for example, a recombinant human arginase I comprising an amino acid sequence of SEQ ID NO:1. In some embodiments, the arginase comprises a catalytic domain of human arginase I comprising an amino acid sequence of SEQ ID NO:2. In some embodiments, the PEGylated-arginase has at least one polyethylene glycol (PEG) molecule that covalently links with an amino acid residue or with more than one amino acid residue of the arginase. In some embodiments, at least one PEG molecule covalently links with a lysine residue or with more than one lysine residue of the arginase. In some embodiments, at least one PEG molecule covalently links with a cysteine residue or with more than one cysteine residue of the arginase. In some embodiments, at least one PEG molecule covalently links with one or more lysine residues, one or more cysteine residues, one or more histidine residues, one or more arginine residues, one or more aspartic acid residues, one or more glutamic acid residues, one or more serine residues, one or more threonine residues, one or more tyrosine residues, an amino (N—) terminal amino group, or a carboxy (C—) terminal carboxylic acid group. In some embodiments, the PEG has a molecular weight of about 5 KDa, about 10 kDa, about 15 kDa, about 20 kDa, about 30 kDa, or about 40 kDa. In some embodiments, the PEG has a molecular weight of from about 10 kDa to about 30 kDa or from about 20 kDa to about 40 kDa.

In some embodiments, the PEGylation of the arginase is achieved by covalently conjugating a PEG molecule with the arginase using a coupling agent. Examples of a coupling agent includes, without limitation, methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-succinimidyl glutarate (mPEG-SG), mPEG-N-hydroxyl-succinimide (mPEG-NHS), mPEG-tresylate, and mPEG-aldehyde. In some embodiments, the coupling agent is methoxy polyethylene glycol-succinimidyl propionate 5000, with an average molecular weight of 5000.

In some embodiments, a PEGylated-arginase disclosed in this application includes a recombinant human arginase, for example, a recombinant human arginase I, in which the recombinant human arginase I has at least one PEG molecule that covalently links with an amino acid residue or with more than one amino acid residue of the recombinant human arginase I. In some embodiments, the PEGylated-arginase, for example, a PEGylated recombinant human arginase I, has about 6-12 PEG molecules per arginase. In some embodiments, the PEG molecule covalently links with a lysine residue or with more than one lysine residue of the PEGylated-arginase, for example, a PEGylated recombinant human arginase I. In some embodiments, the PEG molecule covalently links with a cysteine residue or with more than one cysteine residue of the PEGylated-arginase, for example, a PEGylated recombinant human arginase I. In some embodiments, the PEG molecule covalently links with one or more lysine residues, one or more cysteine residues, one or more histidine residues, one or more arginine residues, one or more aspartic acid residues, one or more glutamic acid residues, one or more serine residues, one or more threonine residues, one or more tyrosine residues, an amino (N—) terminal amino group, or a carboxy (C—) terminal carboxylic acid group of the PEGylated-arginase, for example, a PEGylated recombinant human arginase I.

In some embodiments, an arginase (e.g., a non-PEGylated arginase or PEGylated arginase) described herein includes a terminal protein sequence (for example, an N-terminal or C-terminal protein sequence). A terminal protein sequence can be added, for example, for ease of protein purification or protein identification. Examples, of suitable terminal protein sequences include, but are not limited to, a 6×Histidine (6×His) tag (SEQ ID NO:51), a Flag tag (SEQ ID NO:52), a V5 tag (SEQ ID NO:53), a Myc tag (SEQ ID NO:54), and a Hemagluttinin (HA) tag (SEQ ID NO:55). In some embodiments, a PEGylated arginase described herein, for example, a PEGylated recombinant human arginase described herein, includes a recombinant arginase in which the recombinant arginase has six additional histidine residues at an amino-terminal end thereof, and at least one PEG molecule that covalently links with an amino acid residue or with more than one amino acid residue of the recombinant arginase. In some embodiments, the recombinant arginase has about 6-12 PEG molecules per arginase. In some embodiments, the PEG molecule covalently links with a lysine residue or with more than one lysine residues of the recombinant arginase. In some embodiments, a PEGylated arginase described herein, for example, a PEGylated recombinant human arginase described herein, includes a recombinant arginase in which the recombinant arginase has a 6×His tag, a Flag tag, a V5 tag, a Myc tag, or a HA tag at an amino-terminal end thereof, and at least one PEG molecule that covalently links with an amino acid residue or with more than one amino acid residue of the recombinant arginase. In some embodiments, a PEGylated arginase described herein, for example, a PEGylated recombinant human arginase described herein, includes a recombinant arginase in which the recombinant arginase has a 6×His tag, a Flag tag, a V5 tag, a Myc tag, or a HA tag at a carboxy-terminal end thereof, and at least one PEG molecule that covalently links with an amino acid residue or with more than one amino acid residue of the recombinant arginase.

In some embodiments, a PEGylated arginase described herein, for example, a PEGylated recombinant human arginase I described herein, includes a recombinant human arginase I in which the recombinant arginase has a 6×His tag at an amino-terminal end thereof, and at least one PEG molecule that covalently links with an amino acid residue or with more than one amino acid residue of the recombinant human arginase I. In some embodiments, the recombinant human arginase I has about 6-12 PEG molecules per arginase. In some embodiments, the PEG molecule covalently links with a lysine residue or with more than one lysine residues of the recombinant human arginase I. In some embodiments, a PEGylated recombinant human arginase I described herein includes a 6×His tag, a Flag tag, a V5 tag, a Myc tag, or a HA tag at an amino-terminal end thereof, and at least one PEG molecule that covalently links with an amino acid residue or with more than one amino acid residue of the recombinant human arginase I. In some embodiments, a PEGylated recombinant human arginase I described herein has a 6×His tag, a Flag tag, a V5 tag, a Myc tag, or a HA tag at a carboxy-terminal end thereof, and at least one PEG molecule that covalently links with an amino acid residue or with more than one amino acid residue of the recombinant human arginase I.

As used herein, the term “drug-resistant pathogen” means a pathogen, e.g., a virus, that has developed an ability to resist the effect of a therapeutic, e.g., an antiviral therapeutic, that was capable of treating the pathogen prior to the pathogen developing some feature (e.g., a genetic mutation) that renders it resistant to treatment with the therapeutic. In some embodiments, the drug-resistant pathogen is a drug-resistant virus. In some embodiments, the drug-resistant pathogen is a drug-resistant bacteria. In some embodiments, the drug-resistant pathogen is a drug-resistant fungus. In some embodiments, the drug-resistant pathogen is a drug-resistant amoeba.

As used herein, the term “disease or disorder” means any pathological condition, including but not limited to those caused by a pathogen. In some embodiments, the disease or disorder is a viral disease or disorder, i.e., is caused by a virus. In some embodiments, the disease or disorder is associated with a specific pathogen or type of pathogen. In particular embodiments, the disease or disorder associated with a specific pathogen or type of pathogen is a disease or disorder caused by the specific pathogen or type of pathogen. For example, a virus disease or disorder can be associated with a specific virus, for example, SARS-CoV-2, or a specific group of viruses, for example, a specific genus of viruses, for example, coronaviruses. Similarly, a bacterial disease or disorder can be associated with a specific bacteria or a specific group of bacteria; a fungal disease or disorder can be associated with a specific fungus or a specific group of fungus; and an amoeba disease or disorder can be associated with a specific amoeba or a specific group of amoeba.

As used herein, the term “infection” means invasion and proliferation of pathogens, e.g., viruses, that are not normally present within the host, e.g., a patient. An infection may cause no symptoms and be subclinical, or it may cause symptoms and be clinically apparent. An infection may remain localized, or it may spread, for example, through the blood or lymphatic vessels, to become systemic.

As used herein, the term “prevention” refers to a medication or a treatment designed and used to prevent a disease or disorder from occurring.

As used herein, the term “treat”, “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. preventing the disease from increasing in severity or scope; (c) relieving the disease, i.e. causing partial or complete amelioration of the disease; or (d) preventing relapse of the disease, i.e. preventing the disease from returning to an active state following previous successful treatment of symptoms of the disease or treatment of the disease.

“Individual,” “patient,” or “subject” are used interchangeably herein, and include any animal, e.g. mammals, e.g. mice, rats, other rodents, rabbits, dogs, cats, swine, cattle, sheep, horses, or primates, and most preferably humans. The non-PEGylated arginase or PEGylated arginase compounds and compositions thereof disclosed herein can be administered to a mammal, such as a human. The non-PEGylated arginase or PEGylated arginase compounds disclosed herein can be administered to other mammals, such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). A patient may be an individual diagnosed with a high risk of developing a disease or disorder, for example, an infectious disease or disorder (e.g., an immunocompromised individual), someone who has been diagnosed with a disease or disorder, for example, an infectious disease or disorder, someone who previously suffered from a disease or disorder, for example, an infectious disease or disorder, or an individual evaluated for symptoms or indications of a disease or disorder, for example, an infectious disease or disorder.

The term “patient in need,” as used herein, refers to a patient suffering from any of the symptoms or manifestations of a disease or disorder, for example, an infectious disease or disorder, a patient who may suffer from any of the symptoms or manifestations of a disease or disorder, for example, an infectious disease or disorder, or any patient who might benefit from a method of the disclosure for treating or preventing a disease or disorder, for example, an infectious disease or disorder. A patient in need may include a patient who is diagnosed with a risk of developing a disease or disorder (for example, an infectious disease or disorder), a patient who has suffered from a disease or disorder (for example, an infectious disease or disorder) in the past, or a patient who has previously been treated for a disease or disorder (for example, an infectious disease or disorder).

As used herein, the term “pharmaceutically acceptable composition” means a composition comprising at least one compound, e.g., a non-PEGylated arginase or a PEGylated arginase described herein, formulated together with one or more pharmaceutically acceptable carriers.

As used herein, the term “pharmaceutically acceptable salt” refers to salts of acidic or basic groups that may be present in compounds, for example, arginase compounds (e.g., non-PEGylated or PEGylated arginase compounds), used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

As used herein, the term “pharmaceutically acceptable excipient” means a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with IL-34 inhibitors of the invention. For examples of excipients, see Martin, Remington's Pharmaceutical Sciences, 15th Ed., Mack Publ. Co., Easton, Pa. (1975).

As used herein, the term “therapeutically effective amount,” “effective amount,” or a “pharmaceutically effective amount,” as used herein, refers to the amount of an agent, for example, a non-PEGylated arginase or a PEGylated arginase described herein, that is sufficient to at least partially treat a condition when administered to a patient. The therapeutically effective amount will vary depending on the severity of the condition, the route of administration of the component, and the age, weight, etc. of the patient being treated. Accordingly, an effective amount of a disclosed non-PEGylated arginase or PEGylated arginase is the amount of the non-PEGylated arginase or PEGylated arginase necessary to treat a disease or disorder, for example, an infectious disease or disorder in a patient such that administration of the agent prevents the disease or disorder from occurring in a subject, prevents the disease or disorder progression, or relieves or completely ameliorates some or all associated symptoms of the disease or disorder, e.g., causes clearance of the infection.

As used herein, the term “administering” refers to administration by any suitable route, for example, oral administration, administration as a suppository, topical contact, intravenous, parenteral, intraperitoneal, intramuscular, intralesional, intrathecal, intracranial, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, intrathecal, and intracranial. In some embodiments, methods described herein can include administering by inhalation. For example, in some embodiments, methods described herein can include administering of a composition described herein by inhalation through the oral cavity, the nasal cavity, or the oral and nasal cavity of a patient. Administering by inhalation can be achieved using devices known in the art, for example, a nebuliser or inhaler (e.g., a metered dose inhaler or a dry powder inhaler). Other modes of delivery include, but are not limited to, the use of liposomal formulations, intravenous infusion, transdermal patches, etc.

By “co-administer” it is meant that a compound or composition described herein is administered at the same time, just prior to, or just after the administration of one or more additional therapies (e.g., an antiviral agent). The compounds or compositions described herein can be administered alone or can be co-administered to the patient. Co-administration is meant to include simultaneous or sequential administration of the compound or composition individually or in combination (more than one compound or agent). Thus, the preparations can also be combined, when desired, with other active substances (e.g. to reduce metabolic degradation or to provide an additional therapeutic for disease prevention or treatment).

The description above describes multiple aspects and embodiments of the invention. The patent application specifically contemplates all combinations and permutations of the aspects and embodiments.

### II. Arginase (Non-Pegylated and Pegylated-Arginase)

Embodiments of the present disclosure include methods of modulating amino acid concentrations in microenvironments. Embodiments of the present disclosure modulate amino acid concentrations as a therapeutic approach for treating diseases, for example, viral infections. In certain embodiments, amino acid concentrations are modulated to inhibit viral replication and prevent deleterious inflammation. In embodiments of the present invention, an arginase (e.g., a non-PEGylated arginase or a PEGylated arginase) is administered as an antiviral. Embodiments of the present invention may be utilized to treat a broad range of viral diseases or disorder, including viral infections. In certain embodiments of the present disclosure, an arginase (e.g., a non-PEGylated arginase or a PEGylated arginase) may be administered to a patient infected by a virus. An arginase (e.g., a non-PEGylated arginase or a PEGylated arginase) may be administered to deplete arginine in a recipient. Depletion of arginine may inhibit viral replication or viral transmission. Depletion of arginine may decrease inflammatory immune responses in an infected subject. Administering an arginase (e.g., a non-PEGylated arginase or a PEGylated arginase), or a pharmaceutically acceptable salt thereof, or a composition thereof, can be effective to treat or prevent a virus disease or disorder, a bacterial disease or disorder, a fungal disease or disorder, and/or an amoeba disease or disorder. Administering an arginase (e.g., a non-PEGylated arginase or a PEGylated arginase), or a pharmaceutically acceptable salt thereof, or a composition thereof, can be effective to inhibit a specific process associated with a pathogen, for example, a virus. For example, administering an arginase (e.g., a non-PEGylated arginase or a PEGylated arginase), or a pharmaceutically acceptable salt thereof, or a composition thereof, can be effective to inhibit any or all of the following: virus release from a cell, virus transmission, virus genome replication, virus gene expression, or virus assembly.

Arginase is a manganese-containing enzyme and is the final enzyme of the urea cycle. Specifically, arginase catalyzes the conversion of L-arginine into L-ornithine and urea. In most mammals, two isozymes of arginase exist: arginase I, which functions in the urea cycle and is located primarily in the cytoplasm of the liver, and arginase II, which may regulate arginine/ornithine concentrations in the cell. PEGylation is the process of covalent attachment of polyethylene glycol (PEG) polymer chains to another molecule such as a drug or protein. PEGylation may mask an agent from a host's immune system and may provide increased solubility, mobility and longevity to the agent. PEGylated-arginase formulations described herein include a formulation of an arginase I that has been PEGylated.

Coupling of arginase to PEG improves stability and efficacy of the arginase enzyme. For example, while native arginase is cleared from circulation within minutes (Savoca et al., 1984), a single injection of PEG-Arginase MW5000 in rats was sufficient to achieve near complete arginine depletion for approximately 3 days (Cheng et al., 2007).

Arginase is a homo-trimeric enzyme with an α/β fold of a parallel eight-stranded β-sheet surrounded by several helices. The enzyme contains a di-nuclear metal cluster that is integral to the functionality of the enzyme. Native arginase is complexed with Mn²⁺.

In some embodiments, the present disclosure contemplates mutant arginases wherein Mn²⁺ is replaced with another metal. For example, in some embodiments, a PEGylated-arginase described herein is complexed with a cobalt, iron, or zinc ion, rather than a manganese ion. Substitution of the metal cofactor in human arginase can exert a beneficial effect on the rate of hydrolysis of L-arginine and stability under physiological conditions when compared to native human arginase with the native metal cofactor Me. The substitution of Mn²⁺ with other divalent cations can be exploited to shift the pH optimum of the enzyme to a lower value and thus achieve high rates of L-arginine hydrolysis under physiological conditions. Human Arginase proteins have two Mn (II) sites; therefore, either or both sites can be substituted so as to generate a mutated arginase with a non-native metal cofactor. In some embodiments, the metal is cobalt (Co²⁺). In some embodiments, incorporation of Co2+ in the place of Mn²⁺ results in dramatically higher activity at physiological pH. Mutated Arginases useful for binding to cobalt are provided in U.S. Pat. No. 10,098,933. In some embodiments, the metal is zinc (Zn²⁺). In some embodiments, the metal is iron (Fe²⁺).

In some embodiments, the arginase protein comprises at least one amino acid substitution at the metal binding site. The structure of arginase includes an active site cleft containing two Mn²⁺ ions, with the more deeply localized ion designated Mn_{A} coordinated to H101, D124, D128, D232 and bridging hydroxide. The other metal is designated Mn_{B} and is coordinated by H126, D124, D232, D234 and bridging hydroxide (Christianson and Cox, 1999). The residues comprising the metal binding site for the first shell of Arginase I are H101, D124, H126, D128, D232, and D234 and for the second shell are W122, D181, and 5230. Similarly, the residues comprising the metal binding site for the first shell of Arginase II are H120, D143, H145, D147, D251, D253 and for the second shell are W141, D200, 5249. In some embodiments, the arginase is a mutant arginase. In certain embodiments, the arginase is a C303P variant. In some embodiments, the arginase comprises an Fc-domain protein fusion. In some embodiments, long serum persistence improves the use of arginase as a therapeutic.

In some embodiments, the arginase (e.g., a non-PEGylated arginase or a PEGylated arginase) is used to treat a pathogenic disease or disorder, including but not limited to, a virus disease or disorder, a bacterial disease or disorder, a fungal disease or disorder, and an amoeba disease or disorder. In some embodiments, the arginase (e.g., a non-PEGylated arginase or a PEGylated arginase) is used to treat a pathogenic infection, including but not limited to, a viral infection, a bacterial infection, a fungal infection, and an amoebal infection. In some embodiments, the arginase (e.g., a non-PEGylated arginase or a PEGylated arginase) is effective against RNA virus infections and DNA virus infections, including both enveloped and non-enveloped viruses. In some embodiments, the arginase (e.g., a non-PEGylated arginase or a PEGylated arginase) is administered orally or as an inhalant formulation (for example, an oral inhalant formulation, a nasal inhalant formulation, or a nasal inhalant and oral inhalant formulation). In some embodiments, the arginase (e.g., a non-PEGylated arginase or a PEGylated arginase) is incorporated into an injection composition.

Exemplary amino acid sequences of arginases that may, in certain embodiments, be conjugated to PEG to form a PEGylated-arginase of the present invention are provided in Table 1.

TABLE 1
Exemplary arginase sequences

| SEQ | |
|---|---|
| ID | |
| NO: | Description |
| 1 | MSAKSRTIGIIGAPFSKGQPRGGVEEGPTVLRKAGLLEKLKEQECDVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPCISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLACFGLAREGNHKPIDYLNPPK |
| 2 | HSLAIGSISGHARVHPDLGVIWVDAHTDINTPLTTTSGNLHGQPVSFLLKELKG |
| | KIPDVPGFSWVTPCISAKDIVYIGLRDVDPGEHYILKTLGIKYFSMTEVDRLGIG |
| | KVMEETLSYLLGRKKRPIHLSFDVD |
| 3 | MSAKSRTIGIIGAPFSKGQPRGGVEEGPTVLRKAGLLEKLKEQEADVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPCISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLACFGLAREGNHKPIDYLNPPK |
| 4 | MSAKSRTIGIIGAPFSKGQPRGGVEEGPTVLRKAGLLEKLKEQECDVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPAISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLACFGLAREGNHKPIDYLNPPK |
| 5 | MSAKSRTIGIIGAPFSKGQPRGGVEEGPTVLRKAGLLEKLKEQECDVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPCISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLAAFGLAREGNHKPIDYLNPPK |
| 6 | MSAKSRTIGIIGAPFSKGQPRGGVEEGPTVLRKAGLLEKLKEQEADVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPAISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLACFGLAREGNHKPIDYLNPPK |
| 7 | MSAKSRTIGIIGAPFSKGQPRGGVEEGPTVLRKAGLLEKLKEQEADVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPCISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLAAFGLAREGNHKPIDYLNPPK |
| 8 | MSAKSRTIGIIGAPFSKGQPRGGVEEGPTVLRKAGLLEKLKEQECDVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPAISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLAAFGLAREGNHKPIDYLNPPK |
| 9 | MSAKSRTIGIIGAPFSKGQPRGGVEEGPTVLRKAGLLEKLKEQEADVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPAISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLAAFGLAREGNHKPIDYLNPPK |
| 10 | MKPISIIGVPMDLGQTRRGVDMGPSAMRYAGVIERLERLHYDIEDLGDIPIGKA |
| | ERLHEQGDSRLRNLKAVAEANEKLAAAVDQWQRGRFPLVLGGDHSIAIGTLA |
| | GVAKHYERLGVIWYDAHGDVNTAETSPSGNIHGMPLAASLGFGHPALTQIGG |
| | YSPKIKPEHVVLIGVRSLDEGEKKFIREKGIKIYTMHEVDRLGMTRVMEETIAY |
| | LKERTDGVHLSLDLDGLDPSDAPGVGTPVIGGLTYRESHLAMEMLAEAQIITS |
| | AEFVEVNPILDERNKTASVAVALMGSLFGEKLM |
| 11 | MKPISIIGVPMDLGQTRRGVDMGPSAMRYAGVIERLERLHYDIEDLGDIPIGKA |
| | ERLHEQGDSRLRNLKAVAEANEKLAAAVDQWQRGRFPLVLGGDHSIAIGTLA |
| | GVAKHYERLGVIWYDAHGDVNTAETSPSGNIHGMPLAASLGFGHPALTQIGG |
| | YCPKIKPEHVVLIGVRSLDEGEKKFIREKGIKIYTMHEVDRLGMTRVMEETIAY |
| | LKERTDGVHLSLDLDGLDPSDAPGVGTPVIGGLTYRESHLAMEMLAEAQIITS |
| | AEFVEVNPILDERNKTASVAVALMGSLFGEKLMHHHHHH |
| 12 | MSAKSRTIGIIGAPFSKGQPRGGVEEGPTVLRKAGLLEKLKEQESDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPCISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLACFGLAREGNHKPIDYLNPPK |
| 13 | MSAKSRTIGIIGAPFSKGQPRGGVEEGPTVLRKAGLLEKLKEQECDVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPSISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLACFGLAREGNHKPIDYLNPPK |
| 14 | MSAKSRTIGIIGAPFSKGQPRGGVEEGPTVLRKAGLLEKLKEQECDVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPCISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLASFGLAREGNHKPIDYLNPPK |
| 15 | MSAKSRTIGIIGAPFSKGQPRGGVEEGPTVLRKAGLLEKLKEQESDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPSISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLACFGLAREGNHKPIDYLNPPK |
| 16 | MSAKSRTIGIIGAPFSKGQPRGGVEEGPTVLRKAGLLEKLKEQESDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPCISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLASFGLAREGNHKPIDYLNPPK |
| 17 | MSAKSRTIGIIGAPFSKGQPRGGVEEGPTVLRKAGLLEKLKEQECDVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPSISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLASFGLAREGNHKPIDYLNPPK |
| 18 | MSAKSRTIGIIGAPFSKGQPRGGVEEGPTVLRKAGLLEKLKEQESDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPSISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLASFGLAREGNHKPIDYLNPPK |
| 19 | MSAKSRTIGIIGAPFSKGQPRGGVEEGPTVLRKAGLLEKLKEQEADVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPSISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLACFGLAREGNHKPIDYLNPPK |
| 20 | MSAKSRTIGIIGAPFSKGQPRGGVEEGPTVLRKAGLLEKLKEQESDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPAISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLACFGLAREGNHKPIDYLNPPK |
| 21 | MSAKSRTIGIIGAPFSKGQPRGGVEEGPTVLRKAGLLEKLKEQESDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPCISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLAAFGLAREGNHKPIDYLNPPK |
| 22 | MSAKSRTIGIIGAPFSKGQPRGGVEEGPTVLRKAGLLEKLKEQEADVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPCISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLASFGLAREGNHKPIDYLNPPK |
| 23 | MSAKSRTIGIIGAPFSKGQPRGGVEEGPTVLRKAGLLEKLKEQECDVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPAISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLASFGLAREGNHKPIDYLNPPK |
| 24 | MSAKSRTIGIIGAPFSKGQPRGGVEEGPTVLRKAGLLEKLKEQECDVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPSISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLAAFGLAREGNHKPIDYLNPPK |
| 25 | MSAKSRTIGIIGAPFSKGQPRGGVEEGPTVLRKAGLLEKLKEQESDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPAISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLAAFGLAREGNHKPIDYLNPPK |
| 26 | MSAKSRTIGIIGAPFSKGQPRGGVEEGPTVLRKAGLLEKLKEQESDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPSISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLAAFGLAREGNHKPIDYLNPPK |
| 27 | MSAKSRTIGIIGAPFSKGQPRGGVEEGPTVLRKAGLLEKLKEQESDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPAISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLASFGLAREGNHKPIDYLNPPK |
| 28 | MSAKSRTIGIIGAPFSKGQPRGGVEEGPTVLRKAGLLEKLKEQEADVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPSISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLAAFGLAREGNHKPIDYLNPPK |
| 29 | MSAKSRTIGIIGAPFSKGQPRGGVEEGPTVLRKAGLLEKLKEQEADVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPSISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLASFGLAREGNHKPIDYLNPPK |
| 30 | MSAKSRTIGIIGAPFSKGQPRGGVEEGPTVLRKAGLLEKLKEQEADVKDYGDL |
| | PFADIPNDSPFQIVKNPRSVGKASEQLAGKVAEVKKNGRISLVLGGDHSLAIGSI |
| | SGHARVHPDLGVIWVDAHTDINTPLTTTSGNLHGQPVSFLLKELKGKIPDVPGF |
| | SWVTPAISAKDIVYIGLRDVDPGEHYILKTLGIKYFSMTEVDRLGIGKVMEETL |
| | SYLLGRKKRPIHLSFDVDGLDPSFTPATGTPVVGGLTYREGLYITEEIYKTGLLS |
| | GLDIMEVNPSLGKTPEEVTRTVNTAVAITLASFGLAREGNHKPIDYLNPPK |
| 31 | MSFKSQSIGIIGAPFSKGQPRGGVEEGPTALRKAGLLEKLKEQECDVKDYGDLC |
| | FADVPNDTPFQIVKNPRSVGKANQQLADVVAEIKKNGRTSLVLGGDHSMAIGS |
| | ISGHARVHPDLCVIWVDAHTDINTPLTTTTGNLHGQPVSFLLKELKEKIPEVPG |
| | LSWVTPCLSAKDIVYIGLRDVDPAEHYILKTLGIKYFSMIEVDKLGIGKVMEEA |
| | FSYLLGRKKRPIHLSFDVDGLDPFFTPATGTPVHGGLSYREGIYITEEIYKTGLLS |
| | GLDIMEVNPSLGKTPEEVTRTVNTAVALVLACFGVAREGNHKPIDYLKPPK |
| 32 | MSFKSQSIGIIGAPFSKGQPRGGVEEGPTALRKAGLLEKLKEQECDVKDYGDLS |
| | FADVPNDTPFQIVKNPRSVGKANQQLADVVAEIKKNGRTSLVLGGDHSMAIGS |
| | ISGHARVHPDLSVIWVDAHTDINTPLTTTTGNLHGQPVSFLLKELKEKIPEVPGL |
| | SWVTPSLSAKDIVYIGLRDVDPAEHYILKTLGIKYFSMIEVDKLGIGKVMEEAF |
| | SYLLGRKKRPIHLSFDVDGLDPFFTPATGTPVHGGLSYREGIYITEEIYKTGLLS |
| | GLDIMEVNPSLGKTPEEVTRTVNTAVALVLASFGVAREGNHKPIDYLKPPK |
| 33 | SAKSRTIGIIGAPFSKGQPRGGVEEGPTVLRKAGLLEKLKEQECDVKDYGDLPF |
| | ADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPSISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLASFGLAREGNHKPIDYLNPPK |
| 34 | XSAKSRTIGIIGAPFSKGQPRGGVEEGPTVLRKAGLLEKLKEQECDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPCISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLACFGLAREGNHKPIDYLNPP, |
| | wherein X is either Methionine or nothing. |
| 35 | SVHSVAVIGAPFSQGQKRKGVEHGPAAIREAGLMKRLSSLGCHLKDFGDLSFT |
| | PVPKDDLYNNLIVNPRSVGLANQELAEVVSRAVSDGYSCVTLGGDHSLAIGTIS |
| | GHARHCPDLCVVWVDAHADINTPLTTSSGNLHGQPVSFLLRELQDKVPQLPGF |
| | SWIKPCISSASIVYIGLRDVDPPEHFILKNYDIQYFSMRDIDRLGIQKVMERTFDL |
| | LIGKRQRPIHLSFDIDAFDPTLAPATGTPVVGGLTYREGMYIAEEIHNTGLLSAL |
| | DLVEVNPQLATSEEEAKTTANLAVDVIASSFGQTREGGHIVYDQLPTPSSPDES |
| | ENQARVRI |
| 36 | XSAKSRTIGIIGAPFSKGQPRGGVEEGPTVLRKAGLLEKLKEQECDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPCISAKDIVYIGLRSVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLACFGLAREGNHKPIDYLNPP, |
| | wherein X is either Methionine or nothing. |
| 37 | XSAKSRTIGIIGAPFSKGQPRGGVEEGPTVLRKAGLLEKLKEQECDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPCISAKDIVYIGLRDVDPGEHYILKTLGIKYFSMTEVDRLGIGKVMEETLS |
| | YLLGRKKRPIHLCFDVDGLDPSFTPATGTPVVGGLTYREGLYITEEIYKTGLLS |
| | GLDIMEVNPSLGKTPEEVTRTVNTAVAITLACFGLAREGNHKPIDYLNPP, |
| | wherein X is either Methionine or nothing. |
| 38 | XSAKSRTIGIIGAPFSKGQPRGGVEEGPTVLRKAGLLEKLKEQECDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPCISAKDIVYIGLRDVDPGEHYILKTLGIKYFSMTEVDRLGIGKVMEETLS |
| | YLLGRKKRPIHLGFDVDGLDPSFTPATGTPVVGGLTYREGLYITEEIYKTGLLS |
| | GLDIMEVNPSLGKTPEEVTRTVNTAVAITLACFGLAREGNHKPIDYLNPP, |
| | wherein X is either Methionine or nothing. |
| 39 | XSAKSRTIGIIGAPFSKGQPRGGVEEGPTVLRKAGLLEKLKEQECDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPCISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLAFFGLAREGNHKPIDYLNPP, |
| | wherein X is either Methionine or nothing. |
| 40 | XSAKSRTIGIIGAPFSKGQPRGGVEEGPTVLRKAGLLEKLKEQECDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPCISAKDIVYIGLRDVDPGEHYILKTLGIKYFSMTEVDRLGIGKVMEETLS |
| | YLLGRKKRPIHLSFDVDGLDPSFTPATGTPVVGGLTYREGLYITEEIYKTGLLSG |
| | LDIMEVNPSLGKTPEEVTRTVNTAVAITLAIFGLAREGNHKPIDYLNPP, wherein |
| | X is either Methionine or nothing. |
| 41 | XSAKSRTIGIIGAPFSKGQPRGGVEEGPTVLRKAGLLEKLKEQECDVKDYGDLP |
| | FADIPNDSPFQIVKNPRSVGKASEQLAGKVAEVKKNGRISLVLGGDHSLAIGSIS |
| | GHARVHPDLGVIWVDAHTDINTPLTTTSGNLHGQPVSFLLKELKGKIPDVPGFS |
| | WVTPCISAKDIVYIGLREVDPGEHYILKTLGIKYFSMTEVDRLGIGKVMEETLS |
| | YLLGRKKRPIHLAFDVDGLDPSFTPATGTPVVGGLTYREGLYITEEIYKTGLLS |
| | GLDIMEVNPSLGKTPEEVTRTVNTAVAITLACFGLAREGNHKPIDYLNPP, |
| | wherein X is either Methionine or nothing. |
| 42 | FPLVLGGDHSIAIGTLAGVAKHYERLGVIWYDAHGDVNTAETSPSGNIHGMPL |
| | AASLGFGHPALTQIGGYSPKIKPEHVVLIGVRSLDEGEKKFIREKGIKIYTMHEV |
| | DRLGMTRVMEETIAYLKERTDGVHLSLDLD |
| 43 | HSMAIGSISGHARVHPDLCVIWVDAHTDINTPLTTTTGNLHGQPVSFLLKELKE |
| | KIPEVPGLSWVTPCLSAKDIVYIGLRDVDPAEHYILKTLGIKYFSMIEVDKLGIG |
| | KVMEEAFSYLLGRKKRPIHLSFDVD |
| 44 | MHHHHHHMSAKSRTIGIIGAPFSKGQPRGGVEEGPTVLRKAGLLEKLKEQECD |
| | VKDYGDLPFADIPNDSPFQIVKNPRSVGKASEQLAGKVAEVKKNGRISLVLGG |
| | DHSLAIGSISGHARVHPDLGVIWVDAHTDINTPLTTTSGNLHGQPVSFLLKELK |
| | GKIPDVPGFSWVTPCISAKDIVYIGLRDVDPGEHYILKTLGIKYFSMTEVDRLGI |
| | GKVMEETLSYLLGRKKRPIHLSFDVDGLDPSFTPATGTPVVGGLTYREGLYITE |
| | EIYKTGLLSGLDIMEVNPSLGKTPEEVTRTVNTAVAITLAAFGLAREGNHKPID |
| | YLNPPK |
| 45 | MHHHHHHMSAKSRTIGIIGAPFSKGQPRGGVEEGPTVLRKAGLLEKLKEQECD |
| | VKDYGDLPFADIPNDSPFQIVKNPRSVGKASEQLAGKVAEVKKNGRISLVLGG |
| | DHSLAIGSISGHARVHPDLGVIWVDAHTDINTPLTTTSGNLHGQPVSFLLKELK |
| | GKIPDVPGFSWVTPAISAKDIVYIGLRDVDPGEHYILKTLGIKYFSMTEVDRLGI |
| | GKVMEETLSYLLGRKKRPIHLSFDVDGLDPSFTPATGTPVVGGLTYREGLYITE |
| | EIYKTGLLSGLDIMEVNPSLGKTPEEVTRTVNTAVAITLACFGLAREGNHKPID |
| | YLNPPK |
| 46 | MHHHHHHMSAKSRTIGIIGAPFSKGQPRGGVEEGPTVLRKAGLLEKLKEQEAD |
| | VKDYGDLPFADIPNDSPFQIVKNPRSVGKASEQLAGKVAEVKKNGRISLVLGG |
| | DHSLAIGSISGHARVHPDLGVIWVDAHTDINTPLTTTSGNLHGQPVSFLLKELK |
| | GKIPDVPGFSWVTPCISAKDIVYIGLRDVDPGEHYILKTLGIKYFSMTEVDRLGI |
| | GKVMEETLSYLLGRKKRPIFILSFDVDGLDPSFTPATGTPWGGLTYREGLYITE |
| | EIYKTGLLSGLDIMEVNPSLGKTPEEVTRTVNTAVAITLACFGLAREGNHKPID |
| | YLNPPK |
| 47 | MHHHHHHMSAKSRTIGIIGAPFSKGQPRGGVEEGPTVLRKAGLLEKLKEQECD |
| | VKDYGDLPFADIPNDSPFQIVKNPRSVGKASEQLAGKVAEVKKNGRISLVLGG |
| | DHSLAIGSISGHARVHPDLGVIWVDAHTDINTPLTTTSGNLHGQPVSFLLKELK |
| | GKIPDVPGFSWVTPAISAKDIVYIGLRDVDPGEHYILKTLGIKYFSMTEVDRLGI |
| | GKVMEETLSYLLGRKKRPIHLSFDVDGLDPSFTPATGTPVVGGLTYREGLYITE |
| | EIYKTGLLSGLDIMEVNPSLGKTPEEVTRTVNTAVAITLAAFGLAREGNHKPID |
| | YLNPPK |
| 48 | MHHHHHHMSAKSRTIGIIGAPFSKGQPRGGVEEGPTVLRKAGLLEKLKEQEAD |
| | VKDYGDLPFADIPNDSPFQIVKNPRSVGKASEQLAGKVAEVKKNGRISLVLGG |
| | DHSLAIGSISGHARVHPDLGVIWVDAHTDINTPLTTTSGNLHGQPVSFLLKELK |
| | GKIPDVPGFSWVTPCISAKDIVYIGLRDVDPGEHYILKTLGIKYFSMTEVDRLGI |
| | GKVMEETLSYLLGRKKRPIHLSFDVDGLDPSFTPATGTPVVGGLTYREGLYITE |
| | EIYKTGLLSGLDIMEVNPSLGKTPEEVTRTVNTAVAITLAAFGLAREGNHKPID |
| | YLNPPK |
| 49 | MHHHHHHMSAKSRTIGIIGAPFSKGQPRGGVEEGPTVLRKAGLLEKLKEQEAD |
| | VKDYGDLPFADIPNDSPFQIVKNPRSVGKASEQLAGKVAEVKKNGRISLVLGG |
| | DHSLAIGSISGHARVHPDLGVIWVDAHTDINTPLTTTSGNLHGQPVSFLLKELK |
| | GKIPDVPGFSWVTPAISAKDIVYIGLRDVDPGEHYILKTLGIKYFSMTEVDRLG |
| | IGKVMEETLSYLLGRKKRPIHLSFDVDGLDPSFTPATGTPVVGGLTYREGLYIT |
| | EEIYKTGLLSGLDIMEVNPSLGKTPEEVTRTVNTAVAITLACFGLAREGNHKPI |
| | DYLNPPK |
| 50 | MHHHHHHMSAKSRTIGIIGAPFSKGQPRGGVEEGPTVLRKAGLLEKLKEQEAD |
| | VKDYGDLPFADIPNDSPFQIVKNPRSVGKASEQLAGKVAEVKKNGRISLVLGG |
| | DHSLAIGSISGHARVHPDLGVIWVDAHTDINTPLTTTSGNLHGQPVSFLLKELK |
| | GKIPDVPGFSWVTPAISAKDIVYIGLRDVDPGEHYILKTLGIKYFSMTEVDRLGI |
| | GKVMEETLSYLLGRKKRPIHLSFDVDGLDPSFTPATGTPVVGGLTYREGLYITE |
| | EIYKTGLLSGLDIMEVNPSLGKTPEEVTRTVNTAVAITLAAFGLAREGNHKPID |
| | YLNPPK |
| 56 | MHHHHHHMSAKSRTIGIIGAPFSKGQPRGGVEEGPTVLRKAGLLEKLKEQECD |
| | VKDYGDLPFADIPNDSPFQIVKNPRSVGKASEQLAGKVAEVKKNGRISLVLGG |
| | DHSLAIGSISGHARVHPDLGVIWVDAHTDINTPLTTTSGNLHGQPVSFLLKELK |
| | GKIPDVPGFSWVTPCISAKDIVYIGLRDVDPGEHYILKTLGIKYFSMTEVDRLGI |
| | GKVMEETLSYLLGRKKRPIFILSFDVDGLDPSFTPATGTPWGGLTYREGLYITE |
| | EIYKTGLLSGLDIMEVNPSLGKTPEEVTRTVNTAVAITLACFGLAREGNHKPID |
| | YLNPPK |

In some embodiments described herein, the non-PEGylated arginase or PEGylated arginase comprises an arginase amino acid sequence derived from a specific species. For example, in some embodiments, the non-PEGylated arginase or PEGylated arginase comprises an arginase amino acid sequence derived from a Homo sapiens (i.e., human) arginase sequence, a Bacillus caldovelox arginase sequence, or a Sus scrofa arginase sequence. In some embodiments, the non-PEGylated arginase or PEGylated arginase comprises an amino acid sequence of a specific portion of an arginase (for example, an amino acid sequence comprising the enzymatic domain of an arginase sequence, for example, SEQ ID NO:2, 42, or 43). In some embodiments, the non-PEGylated arginase or PEGylated arginase comprises an amino acid sequence comprising the enzymatic domain of a human arginase sequence, for example, SEQ ID NO:2.

In some embodiments, the non-PEGylated arginase or PEGylated arginase comprises an amino acid sequence related to a human arginase. Examples of human arginases include gene, mRNA, and protein sequences described in NCBI Gene ID: 383, including the protein sequences of NCBI Reference Sequence NP_001231367.1, NCBI Reference Sequence NP_000036.2, and NCBI Reference Sequence NP_001355949.1. In some embodiments, the non-PEGylated arginase or PEGylated arginase comprises an amino acid sequence related to SEQ ID NO:1. For example, the non-PEGylated arginase or PEGylated arginase can comprise an arginase sequence that is at least about 90% (e.g., about 90%, about 91%, about 92%, about 93%, about 94%, about 95%, about 96%, about 97%, about 98%, about 99%, or about 100%) identical to SEQ ID NO:1.

In some embodiments, the non-PEGylated arginase or PEGylated arginase comprises an amino acid sequence related to the enzymatic domain of human arginase. In some embodiments, the non-PEGylated arginase or PEGylated arginase comprises an amino acid sequence related to SEQ ID NO:2. For example, the non-PEGylated arginase or PEGylated arginase can comprise an amino acid sequence that is at least about 90% (e.g., about 90%, about 91%, about 92%, about 93%, about 94%, about 95%, about 96%, about 97%, about 98%, about 99%, or about 100%) identical to SEQ ID NO:2.

In some embodiments, the non-PEGylated arginase or PEGylated arginase comprises an amino acid sequence related to a Bacillus caldovelox arginase. An example of a Bacillus caldovelox arginase is the protein sequence of NCBI GenBank Entry: AAB06939.1, and its associated gene and mRNA sequences. In some embodiments, the non-PEGylated arginase or PEGylated arginase comprises an amino acid sequence related to SEQ ID NO:10. For example, the non-PEGylated arginase or PEGylated arginase can comprise an arginase sequence that is at least about 90% (e.g., about 90%, about 91%, about 92%, about 93%, about 94%, about 95%, about 96%, about 97%, about 98%, about 99%, or about 100%) identical to SEQ ID NO:10.

In some embodiments, the non-PEGylated arginase or PEGylated arginase comprises an amino acid sequence related to a Sus scrofa arginase. Examples of Sus scrofa arginases include gene, mRNA, and protein sequences described in NCBI Gene ID: 397115, including the protein sequences of NCBI Reference Sequence XP_020938406.1, NCBI Reference Sequence XP_005659247.1, NCBI Reference Sequence XP_020938398.1, and NCBI Reference Sequence XP_0209384041 In some embodiments, the non-PEGylated arginase or PEGylated arginase comprises an amino acid sequence related to SEQ ID NO:31. For example, the non-PEGylated arginase or PEGylated arginase can comprise an arginase sequence that is at least about 90% (e.g., about 90%, about 91%, about 92%, about 93%, about 94%, about 95%, about 96%, about 97%, about 98%, about 99%, or about 100%) identical to SEQ ID NO:31.

In some embodiments, a recombinant arginase, or a functional fragment thereof (for example, an enzymatic domain of an arginase protein), can be expressed/produced, e.g., in vivo from bacterial cells, insect cells, mammalian cells, synthetic cells, or in vitro from cell-free systems or chemical synthesis. A recombinant arginase I can be coded by any combination of codons in the degenerate code. In some embodiments, nucleotides are replaced by utilizing the genetic code such that a codon is changed to a different codon that codes for the same amino acid residue. In some embodiments, altering the identity of a cysteine residue of an arginase sequence described in Table 1 can result in a reduction of protein aggregation in solution of: about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, altering the identity of a cysteine residue of an arginase sequence described in Table 1 can result in no greater than 1% aggregation, no greater than 2% aggregation, no greater than 5% aggregation, no greater than 10% aggregation, no greater than 15% aggregation, no greater than 20% aggregation, no greater than 25% aggregation, no greater than 30% aggregation, no greater than 35% aggregation, no greater than 40% aggregation, no greater than 45% aggregation, no greater than 50% aggregation, no greater than 55% aggregation, no greater than 60% aggregation, no greater than 65% aggregation, no greater than 70% aggregation, no greater than 75% aggregation, no greater than 80% aggregation, no greater than 85% aggregation, no greater than 90% aggregation, or no greater than 95% aggregation of arginase protein in solution.

In some embodiments, altering the identity of one or more amino acids of an arginase protein sequence can reduce the aggregation profile of a recombinant arginase I in solution. In some cases, a recombinant arginase I, or a functional fragment thereof, comprises 1 amino acid mutation, 2 amino acid mutations, 3 amino acid mutations, 4 amino acid mutations, 5 amino acid mutations, 6 amino acid mutations, 7 amino acid mutations, 8 amino acid mutations, 9 amino acid mutations, 10 amino acid mutations, 11 amino acid mutations, 12 amino acid mutations, 13 amino acid mutations, 14 amino acid mutations, 15 amino acid mutations, 16 amino acid mutations, 17 amino acid mutations, 18 amino acid mutations, 19 amino acid mutations, 20 amino acid mutations, 21 amino acid mutations, 22 amino acid mutations, 23 amino acid mutations, 24 amino acid mutations, 25 amino acid mutations, 26 amino acid mutations, 27 amino acid mutations, 28 amino acid mutations, 29 amino acid mutations, 30 amino acid mutations, 31 amino acid mutations, 32 amino acid mutations, 33 amino acid mutations, 34 amino acid mutations, 35 amino acid mutations, 36 amino acid mutations, 37 amino acid mutations, 38 amino acid mutations, 39 amino acid mutations, 40 amino acid mutations, 41 amino acid mutations, 42 amino acid mutations, 43 amino acid mutations, 44 amino acid mutations, 45 amino acid mutations, 46 amino acid mutations, 47 amino acid mutations, 48 amino acid mutations, 49 amino acid mutations, or 50 amino acid mutations. In some embodiments, the amino acid mutated is a cysteine. In some embodiments, the amino acid mutation is a cysteine to phenylalanine (C→4) mutation, a cysteine to serine (C→6) mutation, a cysteine to isoleucine (C→I) mutation, or a cysteine to alanine (C→A) mutation.

In some embodiments, an arginase protein sequence can include, but is not limited to, one or more of the following mutations: a cysteine to phenylalanine (C→F) mutation, a cysteine to serine (C→S) mutation, a cysteine to isoleucine (C→I) mutation, a cysteine to alanine mutation, an aspartic acid to glutamic acid (D→E) mutation, an aspartic acid to serine (D→S) mutation, a serine to cysteine (S→C) mutation, a serine to alanine (S→A) mutation, or a serine to glycine (S→G) mutation.

In some embodiments, a recombinant arginase I can have a molecular tag (alternatively referred to herein as “epitope tags”) engineered into the recombinant nucleic acid sequence. In some embodiments, a molecular tag comprises an amino acid sequence, for example, a 6×His tag, Flag tag, V5 tag, myc tag, a glutathione-S-transferase (GST) tag, a maltose binding protein (MBP) tag, a chitin binding protein (CBP) tag, or a hemagglutinin (HA) tag amino acid sequence. A molecular tag can facilitate purification of a recombinant arginase from a crude expression system. In some embodiments, an arginase described herein comprises the amino acid sequence of any one of SEQ ID NOs:1-43 and a molecular tag amino acid sequence (for example, a 6×His tag (e.g., a 6 histidine tag with an N-terminal methionine), Flag tag, V5 tag, myc tag, GST tag, MBP tag, CBP tag, or HA tag amino acid sequence), wherein the molecular tag sequence is proximal to the N-terminus or the C-terminus of the amino acid sequence of SEQ ID NO:1-43. In some embodiments, a PEGylated arginase described herein comprises at least one PEG molecule conjugated to a protein comprising the amino acid sequence of any one of SEQ ID NOs:1-43 and a molecular tag amino acid sequence (for example, a 6×His tag (e.g., a 6 histidine tag with an N-terminal methionine), Flag tag, V5 tag, myc tag, GST tag, MBP tag, CBP tag, or HA tag amino acid sequence), wherein the molecular tag sequence is proximal to the N-terminus or the C-terminus of the amino acid sequence of SEQ ID NO:1-43.

SEQ ID NO: 56 is an arginase protein sequence comprising SEQ ID NO:1 and a 6×His tag with a methionine at the N-terminus. In certain embodiments, the non-PEGylated arginase or PEGylated arginase comprises an arginase sequence that is at least about 90% (e.g., at least about 90%, at least about 91%, at least about 92%, at least about 93%, at least about 94%, at least about 95%, at least about 96%, at least about 97%, at least about 98%, at least about 99%, or about 100%) identical to SEQ ID NO:56.

SEQ ID NOs:44-50 are arginase protein sequences that include one or more amino acid mutations relative to SEQ ID NO:1 and which include an N-terminal polyhistidine (6×His) tag protein sequence. SEQ ID NO: 44 comprises a polyhistidine tag and a C303→A303 mutation relative to SEQ ID NO:1. SEQ ID NO: 45 comprises a polyhistidine tag and a C168→A168 mutation relative to SEQ ID NO:1. SEQ ID NO: 46 comprises a polyhistidine tag and a C45→A45 mutation relative to SEQ ID NO:1. SEQ ID NO: 47 comprises a polyhistidine tag, the C303→A303, and the C168→A168 double mutations relative to SEQ ID NO:1. SEQ ID NO: 48 comprises a polyhistidine tag, the C303→A303 and the C45→A45 double mutations relative to SEQ ID NO:1. SEQ ID NO: 49 comprises a polyhistidine tag, the C168→A168 and the C45→A45 double mutations relative to SEQ ID NO:1. SEQ ID NO: 50 comprises a polyhistidine tag, the C303→A303, the C168→A168, and the C45→A45 triple mutations relative to SEQ ID NO:1.

Methods described herein can include administering a therapeutically effective amount of a arginase comprising the amino acid sequence of SEQ ID NO:44-50, or a pharmaceutically acceptable salt thereof. In some embodiments, methods described herein can include administering a therapeutically effective amount of a composition comprising an arginase comprising the amino acid sequence of SEQ ID NO:44-50, or a pharmaceutically acceptable salt thereof.

Methods described herein can include administering a therapeutically effective amount of a PEGylated-arginase comprising at least one polyethylene glycol molecule conjugated to an arginase comprising the amino acid sequence of SEQ ID NO:44-50, or a pharmaceutically acceptable salt thereof. In some embodiments, methods described herein can include administering a therapeutically effective amount of a composition comprising a PEGylated-arginase, wherein the PEGylated arginase comprises at least one polyethylene glycol molecule conjugated to an arginase comprising the amino acid sequence of SEQ ID NO:44-50, or a pharmaceutically acceptable salt thereof.

Molecular tags are known in the art. Exemplary amino acid sequences of epitope tags are shown in Table 2.

TABLE 2
Exemplary molecular tags

| SEQ ID NO: | Description | SEQUENCE |
|---|---|---|
| 51# | 6X His Tag | HHHHHH |
| 52 | Flag Tag | DYKDDDDK |
| 53 | V5 Tag | GKPIPNPLLGLDST |
| 54 | Myc | EQKLISEEDL |
| 55 | HA Tag | YPYDVPDYA |

The methods described herein can include administration of a PEGylated arginase, for example, administration to a patient in need of treatment. Polyethylene glycols (PEG) are polyethers that include a (—O—CH2-CH2-) backbone. In general, PEGs are compounds of the following general formula:

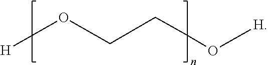

PEG is highly soluble in water, exhibits low immunogenicity, and is non-toxic (see Herzberger et al. (2016) Polymerization of Ethylene Oxide, Propylene Oxide, and Other Alkylene Oxides: Synthesis, Novel Polymer Architectures, and Bioconjugation. Chemical Reviews: 116, 2170-2243). PEGylation can improve pharmacokinetics of the arginase, resulting in sustained duration, improved safety (e.g. lower toxicity, immunogenicity, and antigenicity), increased efficacy, decreased dosing frequency, improved drug solubility and stability, reduced proteolysis, and controlled drug release (Roberts et al., 2002, Adv Drug Deliv Rev, 54:459-76). In some embodiments, PEG helps to increase the half-life of the arginase.

As described herein, an arginase, or a functional fragment thereof (for example, an enzymatic domain of an arginase protein), can be modified with various types of PEG molecules. In some embodiments, a PEG oligomer is methoxy poly(ethylene glycol) succinimidyl proprionate (mPEG-SPA). In some embodiments, a PEG oligomer is a methoxy poly(ethylene glycol) propionic acid (mPEG-acid). In some cases, the disclosure provides a pharmaceutical composition comprising, a purified recombinant human arginase I protein conjugated to at least one polyethylene glycol oligomer. In some cases, the PEGylated recombinant human arginase I protein is conjugated to at least two polyethylene glycol oligomers. In some cases the polyethylene glycol oligomer weighs from about 20 kilodaltons to about 40 kilodaltons. In some cases, the PEGylated recombinant human arginase I protein is conjugated to from about 4 polyethylene glycol molecules to about 13 polyethylene glycol molecules. In some cases the polyethylene glycol oligomer weighs about 5 kilodaltons.

The covalent attachment of an arginase, or a functional fragment thereof, to a polymer polyethylene glycol of interest can change the physicochemical characteristics of the arginase. Examples of physicochemical characteristics that can be altered by binding to a PEG include immunogenicity, in vitro and in vivo biological activity, absorption rate and bioavailability, biodistribution, pharmacokinetic (PK) and pharmacodynamic profiles (PD), and toxicity. In some embodiments, a PEGylated arginase has a reduced immunogenicity. —NH₂, —COOH, —OH, —SH, and disulfide bonds are examples of chemical groups in the amino acid side chain of an arginase that could react with a PEG oligomer. In some embodiments, the amine in the N-terminus and the carboxyl group in the C-terminus can also react with a PEG oligomer.

PEG reagents for protein PEGylation can be activated PEGs. Activated PEGs can be used for amine PEGylation, thiol PEGylation, or N-terminal PEGylation. PEG reagents are commercially available in different lengths, shapes and chemistry allowing them to react with particular functional groups of proteins for their covalent attachment. Non-limiting examples of commercial suppliers of PEG include NOF Corporation (Japan); SunBio (South Korea); Chirotech Technology Limited (UK); JenKem (China); Creative PEGWorks (USA), Sigma-Aldrich (Milwaukee, Wis.), Dendritech (Midland, Mich.), or Polysciences™ (Warrington, Pa.).

Non-limiting examples of commercially available PEGs suitable for use in the embodiments described herein include, but are not limited to, those available from Nektar Therapeutics (San Carlos, Calif.), such as mPEG-NH₂ (Mw about 10 kDa, about 20 kDa), methoxy PEG Succinimidyl α-Methylbutanoate (SMB), SMB-PEG-SMB, methoxy PEG Succinimidyl Propionate (mPEG-SPA), Branched PEG N-Hydroxysuccinimide (mPEG2-NHS), mPEG-CM-HBA-NHS, NHS-HBA-CM-PEG-CM-HBA-NHS, mPEG-ButyrALD, ButyrALD-PEG-ButyrALD, Branched PEG ButyrALD (mPEG2-ButyrALD), Ortho-pyridylthioester (mPEG-OPTE), mPEG Maleimide (MAL), MAL-PEG-MAL, Branched PEG Maleimide (mPEG2-MAL), Forked Maleimide (mPEG-MAL2 and mPEG2-MAL2), mPEG-Ortho-pyridyldisulfide (mPEG-OPSS), OPSS-PEG-OPSS, mPEG-SH, SH-PEG-SH, Amine-PEG-Acid, Boc-PEG-NHS, Fmoc-PEG-NHS, MAL-PEG-NHS, Vinylsulfone-PEG-NHS, and Acrylate-PEG-NHS Ester.

Non-limiting examples of PEGs that can be used in amine pegylation include, for example, PEGs manufactured by Jenken Technology (USA) such as: Y-shape PEG NHS Esters, Y-shape PEG Carboxyl, Glucose PEG NHS Ester, Galactose PEG NHS Ester, Methoxy PEG Succinimidyl Carboxymethyl Ester, Methoxy PEG Carboxyl, Methoxy PEG Succinimidyl Butanoate, Methoxy PEG Succinimidyl Hexanoate, Methoxy PEG Hexanoic Acid, Methoxy PEG Succinimidyl Succinamide, Methoxy PEG Succinimidyl Glutaramide, Methoxy PEG Succinimidyl Carbonate, Methoxy PEG Nitrophenyl Carbonate, Methoxy PEG Succinimidyl Succinate, Methoxy PEG Succinimidyl Glutarate. Non-limiting examples of PEGs that can be used in thiol pegylation include Y-shape PEG Maleimide, Methoxy PEG Maleimide, Methoxy PEG Vinylsulfone, Methoxy PEG Thiol. Non-limiting examples of PEGs that can be used in N-terminal pegylation include, for example, PEGs manufactured by Jenken Technology USA such as: Y-shape PEG Aldehyde, Y-shape PEG Acetaldehyde, Y-shape PEG Propionaldehyde, and Methoxy PEG Propionaldehyde.

In some embodiments, arginase I, or a functional fragment thereof, can have a molecular weight that is smaller than the PEG oligomer to which it is attached. The molecular weight of a PEG oligomer can be, for example, no greater than 100 kilodaltons (kDa), no greater than 95 kDa, no greater than 90 kDa, no greater than 85 kDa, no greater than 80 kDa, no greater than 75 kDa, no greater than 70 kDa, no greater than 65 kDa, no greater than 60 kDa, no greater than 55 kDa, no greater than 50 kDa, no greater than 45 kDa, no greater than 40 kDa, no greater than 35 kDa, no greater than 30 kDa, no greater than 25 kDa, no greater than 20 kDa, no greater than 15 kDa, no greater than 10 kDa, no greater than 5 kDa, no greater than 1 kDa, or no greater than 500 daltons (Da).

In some embodiments, the molecular weight of a PEG molecule can be greater than 500 Da, greater than 1 kilodalton (kDa), greater than 5 kDa, greater than 10 kDa, greater than 15 kDa, greater than 20 kDa, greater than 25 kDa, greater than 30 kDa, greater than 35 kDa, greater than 40 kDa, greater than 45 kDa, greater than 50 kDa, greater than 55 kDa, greater than 60 kDa, greater than 65 kDa, greater than 70 kDa, greater than 75 kDa, greater than 80 kDa, greater than 85 kDa, greater than 90 kDa, greater than 95 kDa, greater than 100 kDa.

In some embodiments, the molecular weight of a PEG oligomer can be from about 1 kDa to about 5 kDa, from about 1 kDa to about 10 kDa, from about 10 kDa to about 20 kDa, from about 10 kDa to about 30 kDa, from about 10 kDa to about 40 kDa, from about 10 kDa to about 50 kDa, from about 20 kDa to about 30 kDa, from about 20 kDa to about 40 kDa, from about 20 kDa to about 50 kDa, from about 30 kDa to about 40 kDa, from about 30 kDa to about 50 kDa.

In certain embodiments, the molecular weight of a PEG oligomer is about 5 kDa. In certain embodiments, the molecular weight of a PEG oligomer is from about 20 kDa to about 40 kDa.

### III. Therapeutic Applications

In certain embodiments, the disease or disorder is the result of an infection by a pathogen, e.g., a bacterium, a virus, a fungus, a protozoan (e.g., an amoeba), an alga, or a prion. Intracellular pathogens include facultative intracellular parasites, which are capable of living and reproducing either inside or outside host cells, and obligate intracellular parasites, which cannot reproduce outside their host cell. In certain embodiments, the intracellular pathogen is a causative agent in a disease or disorder. In certain embodiments, the intracellular pathogen is dormant, latent, or symbiotic within a cell, but can cause a disease or disorder at a later stage of the pathogen's life cycle. An infection by the intracellular pathogen can be acute or chronic. In certain embodiments, the disease or disorder mediated by an intracellular pathogen is a chronic infection. In certain embodiments, the disease or disorder mediated by an intracellular pathogen is an acute infection.

### Treatment of Viral Diseases and Disorders

In certain embodiments, the disease or disorder is caused by a virus. In certain embodiments, the virus is selected from the group consisting of a retrovirus (e.g., human immunodeficiency virus (HIV), simian immunodeficiency virus (SIV), human T-cell lymphotropic virus (HTLV)-1, HTLV-2, HTLV-3, HTLV-4), Ebola virus, hepatitis A virus, hepatitis B virus, hepatitis C virus, a herpes simplex virus (HSV) (e.g., HSV-1, HSV-2, varicella zoster virus, cytomegalovirus), an adenovirus, an orthomyxovirus (e.g., influenza virus A, influenza virus B, influenza virus C, influenza virus D, thogotovirus), a flavivirus (e.g., dengue virus, Zika virus), West Nile virus, Rift Valley fever virus, an arenavirus, Crimean-Congo hemorrhagic fever virus, an echovirus, a rhinovirus, coxsackie virus, a coronavirus (e.g., severe acute respiratory syndrome coronavirus (SARS-CoV), severe acute respiratory syndrome coronavirus 2 (SARS-CoV-2), and Middle East respiratory syndrome—related coronavirus (MERS-CoV)), a respiratory syncytial virus, a mumps virus, a rotavirus, measles virus, rubella virus, a parvovirus (e.g., an adeno-associated virus), a vaccinia virus, a variola virus, a molluscum virus, bovine leukemia virus, a poliovirus, a rabies virus, a polyomavirus (e.g., JC virus, BK virus), an alphavirus, and a rubivirus (e.g., rubella virus). In certain embodiments, the disease or disorder is caused by a virus other than hepatitis and/or HIV.

In certain embodiments, a non-PEGylated arginase or a PEGylated arginase of the described herein is used for treating a disease or disorder caused by a viral infection, e.g., a disease or disorder selected from the group consisting of acquired immune deficiency syndrome (AIDS), HTLV-1 associated myelopathy/tropical spastic paraparesis, Ebola virus disease, hepatitis A, hepatitis B, hepatitis C, herpes, herpes zoster, acute varicella, mononucleosis, respiratory infections, pneumonia, influenza, dengue fever, encephalitis (e.g., Japanese encephalitis), West Nile fever, Rift Valley fever, Crimean-Congo hemorrhagic fever, Kyasanur Forest disease, Yellow fever, Zika fever, aseptic meningitis, myocarditis, common cold, lung infections, molloscum contagiosum, enzootic bovine leucosis, coronavirus disease 2019 (COVID-19), mumps, gastroenteritis, measles, rubella, slapped-cheek disease, smallpox, warts (e.g., genital warts), molluscum contagiosum, polio, rabies, and pityriasis rosea.

In some embodiments, the viral disease or disorder is caused by a human immunodeficiency virus (HIV). HIV refers to two species of retrovirus (HIV-1, HIV-2) that infect cells of the immune system, e.g., CD4⁺ T cells, macrophages, and microglial cells. HIV can progress to acquired immunodeficiency syndrome (AIDS). In some embodiments, the viral disease or disorder is caused by a human papillomavirus (HPV). HPV is a sexually transmitted infection that may result in warts, e.g., genital warts. In some embodiments, the viral disease or disorder is caused by a herpesvirus, e.g., hepatitis C virus (HCV), or cytomegalovirus (CMV). Hepatitis C primarily affects the liver and often leads to liver disease and/or cirrhosis. Cytomegalovirus (CMV), e.g., human cytomegalovirus, is associated with pneumonia and mononucleosis. In some embodiments, the viral disease or disorder is caused by a flavivirus, e.g., Ebola virus, Zika virus, or West Nile virus. Ebola virus causes Ebola virus disease (EVD), a viral hemorrhagic fever.

In some embodiments, the virus is an RNA virus (having a genome that is composed of RNA). RNA viruses may be single-stranded RNA (ssRNA) or double-stranded RNA (dsRNA). RNA viruses have high mutation rates compared to DNA viruses, as RNA polymerase lacks proofreading capability (see Steinhauer DA, Holland J J (1987). “Rapid evolution of RNA viruses”. Annu. Rev. Microbiol. 41: 409-33). Exemplary RNA viruses include, without limitation, bunyaviruses (e.g., hantavirus), coronaviruses (e.g., MERS-CoV, SARS-CoV, SARS-CoV-2), flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses. In some embodiments, the RNA virus is an influenza virus, e.g., influenza A. In some embodiments, the influenza A virus is H1N1 influenza A virus (pandemic H1N1/09). In some embodiments, the influenza A virus is H5N1 influenza A virus (A/Vietnam/2013/04). In some embodiments, the RNA virus is RSV. In some embodiments, the RNA virus is MERS-CoV. In some embodiments, the RNA virus is SARS-CoV2. In some embodiments, the RNA virus is ZIKA.

RNA viruses are classified by the type of genome (double-stranded, negative (−), or positive (+) single-stranded). Double-stranded RNA viruses contain a number of different RNA molecules, each coding for one or more viral proteins. Positive-sense ssRNA viruses utilize their genome directly as mRNA; ribosomes within the host cell translate mRNA into a single protein that is then modified to form the various proteins needed for viral replication. One such protein is RNA-dependent RNA polymerase (RNA replicase), which copies the viral RNA in order to form a double-stranded, replicative form. Negative-sense ssRNA viruses have their genome copied by an RNA replicase enzyme to produce positive-sense RNA for replication. Therefore, the virus comprises an RNA replicase enzyme. The resultant positive-sense RNA then acts as viral mRNA and is translated by the host ribosomes. In some embodiments, the virus is a dsRNA virus. In some embodiments, the virus is a negative ssRNA virus. In some embodiments, the virus is a positive ssRNA virus. In some embodiments, the positive ssRNA virus is a coronavirus.

SARS-CoV2, also sometimes referred to as the novel coronavirus of 2019 or 2019-nCoV, is a positive-sense single-stranded RNA virus. SARS-CoV2 has four structural proteins, known as the S (spike), E (envelope), M (membrane), and N (nucleocapsid) proteins. The N protein holds the RNA genome; together, the S, E, and M proteins form the viral envelope. Spike allows the virus to attach to the membrane of a host cell, such as the ACE2 receptor in human cells. SARS-CoV2 is the highly contagious, causative viral agent of coronavirus disease 2019 (COVID19), a global pandemic.

In some embodiments, the virus is a DNA virus (having a genome that is composed of DNA). Exemplary DNA viruses include, without limitation, parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomaviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). In certain embodiments, the DNA virus is an adenovirus, e.g., AdV5. In certain embodiments, the DNA virus is an enterovirus, e.g., EV71. In certain embodiments, the DNA virus is a herpesvirus, e.g., HSV-1.

In some embodiments, the infection is localized, e.g., to an organ or, e.g., to a tissue. In some embodiments, infection is localized to an organ including but not limited to the eye, the ear, the inner ear, the lungs, trachea, bronchus, bronchioli, the liver, the gall bladder, the bile duct, the kidney, the bladder, the testis, the cervix, the ovary, the uterus, the skin, or the brain. In certain embodiments, the infection is a viral infection (e.g., an HSV-1, an HSV-2, a VZV, a CMV) and is localized to the eye. In certain embodiments, the infection is an adenoviral infection and is localized to the eye. In certain embodiments, the infection is a bacterial infection (e.g., Chlamydia) and is localized to the eye.

In some embodiments, the infection is chronic. As used herein, “chronic” refers to an infection that persists for an extended period of time, or recurs. In some embodiments, the infection is acute. As used herein, “acute” refers to an infection that is of short duration.

Methods to quantify viral replication are known in the art. In some embodiments, viral count is determined using a plaque assay. In some embodiments, viral count is determined using a focus forming assay (FFA). In some embodiments, viral count is determined using an endpoint dilution assay. In some embodiments, viral count is determined using an enzyme-linked immunosorbent assay (ELISA). In some embodiments, viral count is determined using Tunable resistive pulse sensing (TRPS) to detect individual virus particles. In some embodiments, viral replication is determined by quantifying the amount or percentage of host cell death, e.g., in vitro, for example, using propidium iodide (PI) to identify dead cells, quantifying the amount of morphologically rounded cells, or by immunofluorescence microscopy for apoptotic markers. In some embodiments, viral count is determined by measuring viral titer or multiplicity of infection (MOI) or by performing a plaque assay, a focus forming assay, and endpoint dilution assay, a viral protein quantification assay (for example, a hemagglutination assay, a bicinchoninic acid assay (BCA), or a single radial immunodiffusion assay (SRID) assay), transmission electron microscopy analysis, a tunable resistive pulse sensing (TRPS) assay, a flow cytometry assay, a quantitative PCR (qPCR) assay, or an Enzyme-linked immunosorbent assay (ELISA). In some embodiments, viral replication is determined by quantification of viral nucleic acid (for example, viral DNA or viral RNA) content.

Methods to quantify viral transmission are known in the art. In some embodiments, viral transmission is quantified using epidemiological modeling (see, e.g., Graw F. et a, (2016) Modeling Viral Spread. Annu Rev Virol, 3(1)). In some embodiments, viral transmission is assessed in vitro, e.g., in cell culture, e.g., using microscopy, e.g., using transmission electron microscopy (TEM).

Methods to quantify viral assembly are known in the art. In some embodiments, viral assembly is determined using statistical modeling (see, e.g., Clement N et al., (2018) Viral Capsid Assembly: A Quantified Uncertainty Approach. J Comp Biol, 25(1)). In some embodiments, viral assembly is determined using biochemical techniques to determine capsid complex formation, e.g., co-immunoprecipitation, e.g., western blotting. In some embodiments, viral assembly is determined by flow cytometry for detection of colocalized viral protein (see, e.g., Stoffel, C. L. et al. (2005). “Rapid Determination of Baculovirus Titer by a Dual Channel Virus Counter” American Biotechnology Laboratory. 37 (22): 24-25).

Viral genes encode elements necessary for the process of viral infection, a multi-step process, including, for example, attachment to the host cell, penetration, de-envelopment, viral gene transcription cascade, viral protein expression, viral genome replication, viral packaging and assembly, envelopment, transport and maturation, release and egress, and host cell-to-cell transmission. β genes are those genes corresponding to early steps of viral infection, e.g., viral genome replication. γ genes are those genes corresponding to late steps of viral infection, e.g., egress. Methods to quantify viral gene expression are known in the art. In some embodiments, viral gene expression is determined using reverse transcriptase and quantitative polymerase chain reaction (RT-qPCR). In some embodiments, RNA sequencing (RNA-Seq) is used to determine viral gene expression. In some embodiments, viral DNA is quantified using a Southern blot. In some embodiments, β gene expression is quantified. In some embodiments, γ gene expression is quantified. In some embodiments, β gene expression and γ gene expression are quantified. In some embodiments, expression of the entire viral genome is quantified.

Methods to quantify virus release are known in the art. In some embodiments, viral release is determined by biochemical assay, e.g., western blotting, e.g., metabolic labeling (see, e.g., Yadav et al., (2012). “A facile quantitative assay for viral particle genesis reveals cooperativity in virion assembly and saturation of an antiviral protein.” Virology. 429(2): 155-162). In some embodiments, viral release is determined by ELISA. In some embodiments, viral release is determined using electron microscopy, e.g., transmission electron microscopy (TEM). In some embodiments, viral release is determined by infectivity measurements for the detection of virions in a sample, e.g., serum. In some embodiments, viral release is determined by quantification of viral DNA or viral RNA in serum in vivo or culture supernatant in vitro.

In certain embodiments, a non-PEGylated arginase or a PEGylated arginase as described herein is administered in an amount sufficient to reduce one or more of viral replication, viral transmission, viral assembly, viral infection, and viral release in an infected cell, tissue, or subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or by about 100% as compared to an infected cell, tissue, or subject to which the arginase or a PEGylated-arginase is not administered.

### Treatment of Other Diseases and Disorders

In certain embodiments, the disease or disorder is caused by a bacterium, for example, a bacterial infection. In certain embodiments, the bacteria is selected from the group consisting of Chlamydia (e.g., C. trachomatis), Escherichia coli (e.g., enteropathogenic E. coli, enterohemorrhagic E. coli, uropathogenic E. coli, enteroinvasive E. coli), Helicobacter pylori, Mycobacterium (e.g., M. tuberculosis, M. leprae, M. lepromatosis), Listeria (e.g., L. monocytogenes), Shigella (e.g., S. flexneri), Staphylococcus (e.g., S. aureus), Streptococcus (e.g., S. pyogenes), Streptomyces, Pneumococcus, Meningococcus, Gonococcus, Klebsiella (e.g., K pneumoniae), Proteus, Serratia, Pseudomonas (e.g., P. aeruginosa), Legionella, Acinetobacter (e.g., A. baumannii), Corynebacterium (e.g., C. diphtheria), Coxiella (e.g., C. burnetii), Bacillus (e.g., B. anthricis), Bacteroides, Bordetella, Enterococcus (e.g., E. faecalis), Francisella (e.g., F. tularensis), Haemophilus influenza, Neisseria (e.g., N. meningitides, N. gonorrhoeae), Rickettsia, Salmonella (e.g., S. typhimurium), Vibrio cholerae, Clostridium (e.g., C. tetan, C. botulinum), Yersinia (e.g., Y. pestis), Borrielia (e.g., B. burgdorferi), Brucella, Burkholderia, Campylobacter, and Mycoplasma.

In certain embodiments, a non-PEGylated arginase or a PEGylated arginase described herein is used for treating a disease or disorder caused by a bacterium, for example, an intracellular bacterial infection. Methods described herein can be used to treat, for example, a bacterial disease or disorder selected from the group consisting of chlamydia, tuberculosis, peptic ulcers, leprosy, listeriosis, sialadenitis, bacteria-caused diarrhea or food poisoning, strep throat, scarlet fever, impetigo, cellulitis, pneumonia, meningitis, bacterial endocarditis, diverticulitis, disseminated gonococcemia, septic arthritis, gonococcal ophthalmia neonatorum, urinary tract infections, soft tissue infections, spondyloarthropathies (e.g., ankylosing spondylitis), legionellosis (e.g., Legionnaires' disease, Pontiac fever), diphtheria, salmonellosis, anthrax, cholera, tetanus, botulism, fasciitis, gas gangrene, plaque, Lyme disease, brucellosis, melioidosis, Q fever, tularemia, gonorrhea, typhus, mycoplasma pneumonia, gastroenteritis, and walking pneumonia.

In certain embodiments, the disease or disorder is caused by a fungus. In certain embodiments, the fungus is selected from the group consisting of Candida (e.g., C. albicans, C. krusei, C. glabrata, C. tropicalis), Cryptococcus (e.g., C. neoformans, C. gattii), Aspergillus (e.g., A. fumigatus, A. niger), Mucorales (e.g., M. mucor, M. absidia, M. rhizopus), Sporothrix (e.g., S. schenkii), Blastomyces (e.g., B. dermatitidis), Paracoccidioides (e.g., P. brasiliensis), Coccidioides (e.g., C. immitis), Histoplasma (e.g., H. capsulatum), Acremonium, Basidiobolus (e.g., B. ranarum), Cladophialophora (e.g., C. bantiana), Cunninghamella (e.g., C. bertholletiae), Epidermophyton, Exophiala, Exserohilum, Fonsecaea (e.g., F. pedrosoi), Hortaea (e.g., H. werneckii), Lacazia (e.g., L. loboi), Leptosphaeria (e.g., L. maculans), Madurella (e.g., M. mycetomatis), Malassezia, Microsporum, Mucor, Neotestudina, Onychocola, Phialophora, Piedraia, Pneumocystis (e.g., P. jirovecii), Pseudallescheria (e.g., P. boydii), Pyrenochaeta, Rhizomucor, Scedosporium, Scytalidium, Sporothrix, Trichophyton, Trichosporon, and Zygomycete.

In certain embodiments, a non-PEGylated arginase or a PEGylated arginase described herein is used for treating a disease or disorder caused by an intracellular fungal infection, e.g., a disease or disorder selected from the group consisting of candidiasis, cryptococcosis, aspergillosis, mucormycosis, sporotrichosis, blastomycosis, paracoccidioidomycosis, coccidioidomycosis, histoplasmosis, eumycetoma, onychomycosis, hyalohyphomycosis, subcutaneous zygomycosis, cerebral abscesses, phaeohyphomycosis, chromoblastomycosis, mycetoma, pulmonary mucormycosis, tinea corporis, tinea capitis, tinea cruris, tinea pedis, tinea unguium, tinea nigra, Lobo's disease, blackleg disease, mycetoma, pityriasis versicolor, malassezia folliculitis, steroid acne, seborrhoeic dermatitis, neonatal cephalic pustulosis, mucormycosis, maduromycosis, black piedra, pneumocystis pneumonia, pseudallescheriasis, scedosporiosis, sporotrichosis, and zygomycosis.

In certain embodiments, the disease or disorder is caused by a protozoan. In some embodiments, the protozoan is an amoeba. In certain embodiments, the amoeba is selected from the group consisting of Apicomplexans (Plasmodium (e.g., P. vivax, P. falciparum, P. ovale, P. malariae, Toxoplasma gondii, Cryptosporidium parvum, Babesia microti, Cyclospora cayetanensis, Cystoisospora belli), Trypanosoma (e.g., Trypanosoma brucei, Trypanosoma cruzi), and Leishmania (e.g., Leishmania donovani).

In certain embodiments, a non-PEGylated arginase or a PEGylated arginase described herein is used for treating a disease or disorder caused by an intracellular amoebal infection, e.g., a disease or disorder selected from the group consisting of babesiosis, malaria, cryptosporidiosis, cyclosporiasis, cystoisosporiasis, toxoplasmosis, trypanosomiasis, Chagas disease, and leishmaniasis.

In certain embodiments, the infectious disease or disorder is caused by an alga. In certain embodiments, the alga is a Prototheca. In certain embodiments, a non-PEGylated arginase or a PEGylated arginase described herein is used for treating a disease or disorder caused by an intracellular algal infection, e.g., protothecosis.

In certain embodiments, the infectious disease or disorder is caused by a prion. In certain embodiments, a non-PEGylated arginase or a PEGylated arginase described herein is used for treating a disease or disorder caused by an intracellular prion infection, e.g., a disease or disorder selected from the group consisting of Creutzfeldt-Jakob disease, variant Creutzfeldt-Jakob disease, Gerstmann-Straussler-Scheinker syndrome, fatal familial insomnia, and kuru.

### IV. Combination Therapies

Methods of treatment of the present invention can be used as a monotherapy or in combination with one or more other therapies (for example, anti-infective agents) that can be used to treat a disease or disorder, for example, an infection. The term “combination,” as used herein, is understood to mean that two or more different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Accordingly, in certain embodiments, the subject has received, is receiving, or is scheduled to receive one or more other therapies suitable for use in treating the disease or disorder. In certain embodiments, the method of treatment of the present invention further comprises administering to the subject one or more other therapies suitable for use in treating a disease or disorder, for example, an infection. In certain embodiments, the one or more other therapies comprise an agent that ameliorates one or more symptoms of infection with an intracellular pathogen. In certain embodiments, the one or more other therapies comprise surgical removal of an infected tissue.

It is understood that a method of use disclosed herein can be used in combination with an agent, for example, an anti-infective agent that ameliorates one or more symptoms of a disease or disorder associated with an intracellular pathogen. For example, a method of use disclosed herein can be used in combination with an antiviral agent.

Therapies suitable for treating infections by intracellular pathogens are generally known in the art and are reviewed, for example, by Kamaruzzaman et al. (2017) BR. J. PHARMACOL. 174(14): 2225-36 and De Clercq et al. (2016) CLIN. MICROBIOL. REV. 29(3): 695-747. In certain embodiments, the anti-infective agent inhibits or reduces the viability, proliferation, infectivity, and/or virulence of the intracellular pathogen. Intracellular pathogens may evade immune surveillance and challenge by residing in a latent state. Accordingly, in certain embodiments, the anti-infective agent reverses the latency of the intracellular pathogen such that the infection can be recognized by the host's immune system.

In certain embodiments, the intracellular pathogen is a virus, and the anti-infective agent is an antiviral agent. Exemplary antiviral agents that can be used in the combination include but are not limited to abacavir, acyclovir, adefovir, amprenavir, atazanavir, cidofovir, darunavir, delavirdine, didanosine, docosanol, efavirenz, elvitegravir, emtricitabine, enfuvirtide, entecavir, etravirine, famciclovir, foscarnet, fomivirsen, ganciclovir, indinavir, idoxuridine, lamivudine, lopinavir, maraviroc, MK-2048, nelfinavir, nevirapine, penciclovir, raltegravir, rilpivirine, ritonavir, saquinavir, stavudine, tenofovir trifluridine, valaciclovir, valganciclovir, vidarabine, ibacitabine, amantadine, oseltamivir, rimantidine, tipranavir, zalcitabine, zanamivir, peramivir, danoprevir, remdesivir, and zidovudine. In particular, where the intracellular pathogen is an HIV, exemplary anti-HIV agents that can be used in the combination include, but are not limited to, nucleoside/nucleotide reverse transcriptase inhibitors (e.g., lamivudine, abacavir, zidovudine, stavudine, didanosine, emtricitabine, and tenofovir), non-nucleoside reverse transcriptase inhibitors (e.g., delavirdine, efavirenz, etravirine, and nevirapine), protease inhibitors (e.g., amprenavir, fosamprenavir, atazanavir, darunavir, indinavir, lopinavir, ritonavir, nelfinavir, saquinavir, and tipranavir), fusion or entry inhibitors (e.g., enfuvirtide and maraviroc), integrase inhibitors (e.g., raltegravir and cabotegravir), and latency-reversing agents (e.g., HDAC inhibitors (e.g., vorinostat) and TLR7 agonists (e.g., GS-9620, e.g., as described in U.S. Patent Publication No. US20160008374A1)).

In certain embodiments, the intracellular pathogen is a bacterium, and the anti-infective agent is an anti-bacterial agent. Exemplary anti-bacterial agents that can be used in the combination include but are not limited to vancomycin, metronidazole, gentamicin, colistin, fidaxomicin, telavancin, oritavancin, dalbavancin, daptomycin, cephalexin, cefuroxime, cefadroxil, cefazolin, cephalothin, cefaclor, cefamandole, cefoxitin, cefprozil, ceftobiprole, cipro, Levaquin, floxin, tequin, avelox, norflox, tetracycline, minocycline, oxytetracycline, doxycycline, amoxicillin, ampicillin, penicillin V, dicloxacillin, carbenicillin, methicillin, ertapenem, doripenem, imipenem/cilastatin, meropenem, amikacin, kanamycin, neomycin, netilmicin, tobramycin, paromomycin, cefixime, cefdinir, cefditoren, cefoperazone, cefotaxime, ceftazidime, ceftibuten, ceftizoxime, ceftriaxone, cefoxotin, and streptomycin.

In certain embodiments, the intracellular pathogen is a fungus, and the anti-infective agent is an anti-fungal agent. Exemplary anti-fungal agents that can be used in the combination include but are not limited to natamycin, rimocidin, filipin, nystatin, amphotericin B, candicin, and hamycin, miconazole, ketoconazole, clotrimazole, econazole, omoconazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, and albaconazole, abafungin, terbinafine, naftifine, butenafine, anidulafungin, caspofungin, micafungin, polygodial, benzoic acid, ciclopirox, tolnaftate, undecylenic acid, flucytosine or 5-fluorocytosine, griseofulvin, and haloprogin.

In certain embodiments, the intracellular pathogen is a protozoan, and the anti-infective agent is an anti-protozoal agent. Exemplary anti-protozoal agents that can be used in the combination include but are not limited to quinine (optionally in combination with clindamycin), chloroquine, amodiaquine, artemisinin and its derivatives (e.g., artemether, artesunate, dihydroartemisinin, arteether), doxycycline, pyrimethamine, mefloquine, halofantrine, hydroxychloroquine, eflornithine, nitazoxanide, ornidazole, paromomycin, pentamidine, primaquine, pyrimethamine, proguanil (optionally in combination with atovaquone), sulfonamides (e.g., sulfadoxine, sulfamethoxypyridazine), tafenoquine, and tinidazole. In specific embodiments, the intracellular pathogen is a Plasmodium (e.g., P. vivax, P. falciparum, P. ovale, P. malariae), and the anti-infective agent is an anti-malarial agent. Exemplary anti-malarial agents that can be used in the combination include but are not limited to quinine (optionally in combination with clindamycin), chloroquine, amodiaquine, artemisinin and its derivatives (e.g., artemether, artesunate, dihydroartemisinin, arteether), doxycycline, halofantrine, mefloquine, primaquine, proguanil (optionally in combination with atovaquone), sulfonamides (e.g., sulfadoxine, sulfamethoxypyridazine), tafenoquine. It is understood that many of these anti-malarial agents can be used in combination especially for treating severe and/or acute infections.

In certain embodiments, the intracellular pathogen is an alga, and the anti-infective agent is an anti-algal agent. Exemplary anti-algal agents that can be used in the combination include but are not limited to ketoconazole, itraconazole, fluconazole, and voriconazole.

In certain embodiments, the intracellular pathogen is a prion, and the anti-infective agent is an anti-prion agent. Exemplary anti-prion agents that can be used in the combination include but are not limited to pentosan polysulfate, quinacrine, thioflavine, amphotericin B, tetracyclines, tricyclic antidepressants (e.g., desipramine), and lithium chloride.

An additional class of agents that may be used as part of a combination therapy in treating an infectious disease is immunotherapies, e.g., immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3.

Appropriate therapies can be selected according to diagnosis of the specific infection. Wherein the subject is infected with a plurality of pathogens (e.g., a plurality of intracellular pathogens, e.g., a plurality of viral infections), two or more appropriate therapies for treating these infections may be used in combination with a PEGylated-arginase described herein.

### V. Pharmaceutical Compositions and Methods of Delivery

The present disclosure also features pharmaceutical compositions that contain a therapeutically effective amount of a non-PEGylated arginase or a PEGylated arginase described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

### Parenteral Delivery

An intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and 45 mg of the freeze-dried formulation may be contained in one vial. In certain embodiments, the about 40 mg—about 100 mg of freeze-dried formulation may be contained in one vial. In certain embodiments, a freeze-dried formulation from vials, e.g., 12, 27, or 45 vials, are combined to obtain a therapeutic dose of the PEGylated-arginase in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial. The non-PEGylated arginase or PEGylated arginase could exist in a liquid aqueous pharmaceutical formulation including a therapeutically effective amount of the non-PEGylated arginase or PEGylated arginase in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including a non-PEGylated arginase or a PEGylated arginase of the present disclosure, in combination with one or more of mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86 mg/mL), and about 6.2 mg/mL of sodium chloride (e.g., 6.165 mg/mL). In certain embodiments, the buffer system includes about 1 to about 1.5 mg/mL of citric acid, about 0.25 to about 0.5 mg/mL of sodium citrate, about 1.25 to about 1.75 mg/mL of disodium phosphate dihydrate, about 0.7 to about 1.1 mg/mL of sodium dihydrogen phosphate dihydrate, and about 6.0 to about 6.4 mg/mL of sodium chloride. In certain embodiments, the pH of the formulation is adjusted with sodium hydroxide.

A polyol, which acts as a tonicifier, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10 to about 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added can minimize the formation of particulates in the formulation and/or reduce adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, a composition described herein is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments, the liquid formulation may be diluted with 0.9% saline solution. In certain embodiments, a composition described herein may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as a 1 dalton mass increase. Deamidation of an asparagine results in either aspartic acid or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, compositions of the present disclosure may be preserved under conditions of pH and humidity to prevent deamination of the protein product.

Compositions described herein can include an aqueous carrier. Aqueous carriers of interest herein are pharmaceutically acceptable (safe and non-toxic for administration to a human) and are useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution, and dextrose solution.

A preservative may be optionally added to the formulations described herein to reduce bacterial action. The addition of a preservative may, for example, facilitate the production of a multi-use (multiple-dose) formulation.

Intravenous (IV) formulations may be the preferred administration route in particular instances, such as when a patient is in the hospital receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer component may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, or citrate buffers, in which case, sodium, potassium or ammonium ions can serve as counterion.

In certain embodiments, the lyophilized drug product may be constituted with an aqueous carrier. The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution, and dextrose solution.

Arginases (e.g., non-PEGylated or PEGylated arginases) of the present disclosure can exist in a lyophilized formulation including the arginase and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative. The amount of sucrose or maltose useful for stabilization of the lyophilized drug product may be in a weight ratio of at least 1:2 protein to sucrose or maltose. In certain embodiments, the non-PEGylated arginase or PEGylated arginase to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. Lyophilized formulations of the present invention may contain such bulking agents.

In certain embodiments, a lyophilized drug product described herein is reconstituted with either Sterile Water for Injection, USP (SWFI) or 0.9% Sodium Chloride Injection, USP. During reconstitution, the lyophilized powder dissolves into a solution.

In certain embodiments, a lyophilized composition described herein is constituted to about 4.5 mL water for injection and diluted with 0.9% saline solution (sodium chloride solution).

In some embodiments, a non-PEGylated arginase or a PEGylated arginase of the present disclosure is administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In some embodiments, delivery of an arginase (e.g., a non-PEGylated arginase or a PEGylated arginase) as disclosed herein is via an inhalation route. Inhalation may be mediated through an oral and/or nasal cavity. In some embodiments, inhalation is with the aid of a nebulizer or an inhaler (e.g., a metered dose inhaler or a dry powder inhaler). In certain embodiments, delivery is through inhalation of a liquid mist. In certain embodiments, delivery is through inhalation of a solid. In some embodiments, the solid is nanosized and formulated in combination with nanoparticles, nanodiamonds, or nanocarbons, or packaged in liposomes or liposome-based packages.

### Enteral Administration

In some embodiments, contemplated herein are compositions suitable for oral delivery of a disclosed arginase (e.g., a non-PEGylated arginase or PEGylated arginase), for example, tablets that include an enteric coating, e.g., a gastro-resistant coating, such that the compositions may deliver the arginase to, e.g., the gastrointestinal tract of a patient.

For example, a tablet for oral administration is provided that comprises granules (e.g., is at least partially formed from granules) that include a disclosed arginase (e.g., a non-PEGylated arginase or PEGylated arginase) and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed arginase (e.g., a non-PEGylated arginase or PEGylated arginase) and a pharmaceutically acceptable salt, and a pharmaceutically acceptable filler. For example, a disclosed non-PEGylated arginase or PEGylated arginase and a filler may be blended together, optionally, with other excipients, and formed into granules. In some embodiments, the intragranular phase may be formed using wet granulation, e.g. a liquid (e.g., water) is added to the blended non-PEGylated arginase or PEGylated arginase and filler, and then the combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include a intragranular phase and/or a extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated formulations, e.g., that include an intragranular phase and/or an extragranular phase, may include a disintegrant such as but are not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated formulation includes an intra-granular phase comprising a disclosed non-PEGylated arginase or PEGylated arginase and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a contemplated formulation may include a lubricant, e.g. an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, a composition described herein comprises an enteric coating. Generally, enteric coatings create a barrier for the oral medication that controls the location at which the drug is absorbed along the digestive track. Enteric coatings may include a polymer that disintegrates at different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE®, Eudragit® grades. In some embodiments, an enteric coating may comprise about 5% to about 10%, about 5% to about 20%, 8 to about 15%, about 8% to about 20%, about 10% to about 20%, or about 12 to about 20%, or about 18% of a contemplated tablet by weight. For example, enteric coatings may include an ethylacrylate-methacrylic acid copolymer.

### Dosage

Actual dosage levels of the active ingredients in the pharmaceutical compositions described herein may be varied so as to obtain an amount of the active ingredient which is effective to achieve the desired therapeutic response for a particular patient, composition, and mode of administration, without being toxic to the patient.

The specific dose can be a uniform dose for each patient, for example, 1 ng/kg to 1 mg/kg of protein daily. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the arginase in a patient can be measured to see if the dosage needs to be adjusted to reach or maintain an effective concentration. Pharmacogenomics may be used to determine which arginase, and dosages thereof, is most likely to be effective for a given individual (Schmitz et al., Clinica Chimica Acta 308: 43-53, 2001; Steimer et al., Clinica Chimica Acta 308: 33-41, 2001).

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose reclosable containers can be used, for example, in combination with a preservative or without a preservative. In some embodiments, the pharmaceutical composition does not comprise a preservative. Formulations for parenteral injection can be presented in unit dosage form, for example, in ampoules, or in multi-dose containers with a preservative.

An arginase (e.g., a non-PEGylated arginase or a PEGylated arginase) described herein can be present in a composition in a range of from about 1 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

An arginase (e.g., a non-PEGylated arginase or a PEGylated arginase) described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

A therapeutically effective dose of an arginase (e.g., a non-PEGylated arginase or a PEGylated arginase) described herein can be from about 1 ng/kg to about 10 ng/kg, from about 1 ng/kg to about 100 ng/kg, from about 1 ng/kg to about 1 mg/kg, from about 1 ng/kg to about 10 mg/kg, from about 1 ng/kg to about 100 mg/kg, from about 1 ng/kg to about 250 mg/kg, from about 1 ng/kg to about 500 mg/kg, from about 1 ng/kg to about 750 mg/kg, from about 1 ng/kg to about 1,000 mg/kg, from about 1 ng/kg to about 1,250 mg/kg, from about 1 ng/kg to about 1,500 mg/kg, from about 1 ng/kg to about 1,750 mg/kg, from about 1 ng/kg to about 2,000 mg/kg, from about 10 ng/kg to about 100 ng/kg, from about 10 ng/kg to about 1 mg/kg, from about 10 ng/kg to about 10 mg/kg, from about 10 ng/kg to about 100 mg/kg, from about 10 ng/kg to about 500 mg/kg, from about 10 ng/kg to about 750 mg/kg, from about 10 ng/kg to about 1,000 mg/kg, from about 10 ng/kg to about 1,250 mg/kg, from about 10 ng/kg to about 1,500 mg/kg, from about 10 ng/kg to about 2,000 mg/kg, from about 100 ng/kg to about 1 mg/kg, from about 100 ng/kg to about 10 mg/kg, from about 100 ng/kg to about 100 mg/kg, from about 100 ng/kg to about 250 mg/kg, from about 100 ng/kg to about 500 mg/kg, from about 100 ng/kg to about 750 mg/kg, from about 100 ng/kg to about 1,000 mg/kg, from about 100 ng/kg to about 1,250 mg/kg, from about 100 ng/kg to about 1,500 mg/kg, from about 100 ng/kg to about 1,750 mg/kg, from about 100 ng/kg to about 2,000 mg/kg, from about 1 mg/kg to about 10 mg/kg, from about 1 mg/kg to about 100 mg/kg, from about 1 mg/kg to about 500 mg/kg, from about 1 mg/kg to about 750 mg/kg, from about 1 mg/kg to about 1,000 mg/kg, from about 1 mg/kg to about 1,250 mg/kg, from about 1 mg/kg to about 1,500 mg/kg, from about 1 mg/kg to about 1,750 mg/kg, from about 1 mg/kg to about 2,000 mg/kg, from about 10 mg/kg to about 100 mg/kg, from about 10 mg/kg to about 500 mg/kg, from about 10 mg/kg to about 750 mg/kg, from about 10 mg/kg to about 1,000 mg/kg, from about 10 mg/kg to about 1,250 mg/kg, from about 10 mg/kg to about 1,500 mg/kg, from about 10 mg/kg to about 1,750 mg/kg, from about 10 mg/kg to about 2,000 mg/kg, from about 100 mg/kg to about 500 mg/kg, from about 100 mg/kg to about 750 mg/kg, from about 100 mg/kg to about 1,000 mg/kg, from about 100 mg/kg to about 1,250 mg/kg, from about 100 mg/kg to about 1,500 mg/kg, from about 100 mg/kg to about 1,750 mg/kg, from about 100 mg/kg to about 2,000 mg/kg, from about 500 mg/kg to about 750 mg/kg, from about 500 mg/kg to about 1,000 mg/kg, from about 500 mg/kg to about 1,250 mg/kg, from about 500 mg/kg to about 1,500 mg/kg, from about 500 mg/kg to about 1,750 mg/kg, from about 500 mg/kg to about 2,000 mg/kg, from about 750 mg/kg to about 1,000 mg/kg, from about 750 mg/kg to about 1,250 mg/kg, from about 750 mg/kg to about 1,500 mg/kg, from about 750 mg/kg to about 1,750 mg/kg, from about 750 mg/kg to about 2,000 mg/kg, from about 1,000 mg/kg to about 1,250 mg/kg, from about 1,000 mg/kg to about 1,500 mg/kg, from about 1,000 mg/kg to about 1,750 mg/kg, or from about 1,000 mg/kg to about 2,000 mg/kg.

In some embodiments, a therapeutically effective dose of an arginase (e.g., a non-PEGylated arginase or a PEGylated arginase) composition described herein can include about 1 ng, about 10 ng, about 50 ng, about 100 ng, about 200 ng, about 250 ng, about 300 ng, about 400 ng, about 500 ng, about 600 ng, about 700 ng, about 750 ng, about 800 ng, about 900 ng, about 1000 ng, about 10 μg, about 50 μg, about 100 μg, about 200 μg, about 250 μg, about 300 μg, about 400 μg, about 500 μg, about 600 μg, about 700 μg, about 750 μg, about 800 μg, about 900 μg, about 1000 μg, about 5 mg, about 10 mg, about 100 mg, about 200 mg, about 250 mg, about 300 mg, about 400 mg, about 500 mg, about 600 mg, about 700 mg, about 750 mg, about 800 mg, about 900 mg, about 1000 mg, about 1500 mg, about 2000 mg, about 3000 mg, about 4000 mg, about 5000 mg, about 6000 mg, about 7000 mg, about 8000 mg, about 9000 mg, or about 10 g of a non-PEGylated arginase or a PEGylated arginase.

In some embodiments, the therapeutically-effective amount of an arginase (e.g., a non-PEGylated arginase or a PEGylated arginase) is from about 1 mg/kg to about 10 mg/kg. In some embodiments, the therapeutically-effective amount of the arginase is from about 10 mg/kg to about 100 mg/kg. In some embodiments, the therapeutically-effective amount of the arginase is greater than 100 mg/kg.

An arginase (e.g., a non-PEGylated arginase or a PEGylated arginase) (comprising, for example, an arginase protein sequence or a functional fragment thereof, for example a catalytic domain of an arginase sequence) as described herein can be administered before, during, or after the occurrence of a disease or condition, for example, an infectious disease or condition. In some embodiments, the arginase can be used as a prophylactic and can be administered continuously to a subject with a propensity to a condition or disease in order to lessen a likelihood of the occurrence of the disease or condition. The arginase can be administered to a subject during or as soon as possible after the onset of symptoms. The administration of the arginase can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The administration of the arginase of the present disclosure can be for a length of time necessary for the treatment of the disease or disorder, such as, for example, from about 24 hours to about 48 hours, from about 48 hours to about 1 week, from about 1 week to about 2 weeks, from about 2 weeks to about 1 month, from about 1 month to about 3 months. In some embodiments, an arginase can be administered for at least 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years at least 3 years, at least 4 years, at least 5 years, or for life. The length of treatment can vary for each subject.

In certain embodiments, treatment begins when a subject is asymptomatic. In some embodiments, treatment begins when a subject is displaying symptoms. Symptoms of an infectious disease or disorder include, without limitation, fever, chills, congestion, fatigue, muscle aches, headache, sore throat, earache, diarrhea, vomiting, dehydration, shock, cough, dry cough, sneezing, rash, shortness of breath, anosmia, pneumonia, elevated heartrate, low blood pressure, seizure, confusion, delirium, hallucination, tremor, impaired coordination, conjunctivitis, cytokine storm, organ failure, and death.

As used herein, “cytokine storm” refers to an acute overreaction of the immune system also known in the art as cytokine release syndrome or cytokine storm syndrome; such an immune response is a systemic inflammatory response syndrome that can result from an infectious disease or disorder. Cytokine storm pathology is associated with inflammation that begins at a local site and spreads throughout the body, for example, via systemic circulation. Cytokine storm pathology resulting from viral infection is associated with acute lung injury and acute respiratory distress syndrome. Cytokine storm pathology is described, for example, in Tisonick et al., (2012) “Into the Eye of the Cytokine Storm,” Microbiol. Mol. Biol. Rev., 76(1):16-32. In certain embodiments, the infectious disease or disorder is COVID-19 and the symptom is cytokine storm. In certain embodiments, the infectious disease or disorder is COVID-19 and the symptom is pneumonia.

In some embodiments, a method described herein, for example, a method that includes administering an arginase (e.g., a non-PEGylated arginase or a PEGylated arginase), a pharmaceutically acceptable salt thereof, or a composition thereof, is effective to modulate cytokine release. In some embodiments, a method described herein, for example, a method that includes administering an arginase, a pharmaceutically acceptable salt thereof, or a composition thereof, is effective to modulate expression of Interleukin 6 (IL-6) or Interferon gamma (IFNγ). In some embodiments, a method described herein, for example, a method that includes administering an arginase, a pharmaceutically acceptable salt thereof, or a composition thereof, is effective to modulate expression or secretion of tumor necrosis factor (TNF), IL-β, MCP-1, IL-6, IFNγ, IL-10.

The amount administered will depend on variables such as the type and extent of disease or infection to be treated, the overall health and size of the patient, the in vivo potency of the arginase (e.g., a non-PEGylated arginase or a PEGylated arginase), the pharmaceutical formulation, and the route of administration. The initial dosage can be increased in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study. Dosing frequency can vary, depending on factors such as route of administration, dosage amount and the disease being treated. Exemplary dosing frequencies are three times per day, twice per day, once per day, once per every two days, once per every three days, once per every four days, once per every five days, once per every six days, once per week, and once every two weeks.

Administration of an arginase (e.g., a non-PEGylated arginase or a PEGylated arginase) described herein can be, but is not limited to, intravenous, intraarterial, intraperitoneal, intramuscular, subcutaneous, intrapleural, intrathecal, intracavitary, by inhalation, by perfusion through a catheter, or by direct intralesional injection.

The description above describes multiple aspects and embodiments of the methods, compositions, and kits described herein. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and is not intended to limit the invention.

### Example 1: PEGylated Arginase Inhibits Replication of Severe Acute Respiratory Syndrome-Related Coronavirus (SARSr-CoV)

In this example, the effect of PEGylated arginase on the replication of SARSr-CoV strains is determined. The SARSr-CoV strains examined included SARS-CoV and SARS-CoV-2 that caused the 2002-2004 SARS outbreak and COVID-19, respectively.

The PEGylated arginase used in this example was BCT-100, having the sequence of SEQ ID NO: 56 and made as described in U.S. Pat. No. 9,109,218, except that in this example wild-type human arginase 1 with N-terminal 6xHis-tag was used (i.e., SEQ ID NO: 56).

Vero E6 cells were maintained as a monolayer in Eagle's Minimum Essential Medium (MEM) supplemented with 10% fetal bovine serum (Thermo Fisher Scientific, USA) at 37° C. and 5% CO₂. The cells were resuspended in fresh complete growth medium at a density of 2×10⁵ cells/mL and seeded into a 24-well culture plate. The cells were treated with varying doses of PEGylated arginase and infected with SARS-CoV or SARS-CoV-2 at multiplicity of infection (MOI) of 0.01. Culture supernatants were collected 48 hours-post-infection. Viral load of SARS-CoV and SARS-CoV-2 in the collected supernatants was quantified using median tissue culture infectious dose (TCID₅₀) assay.

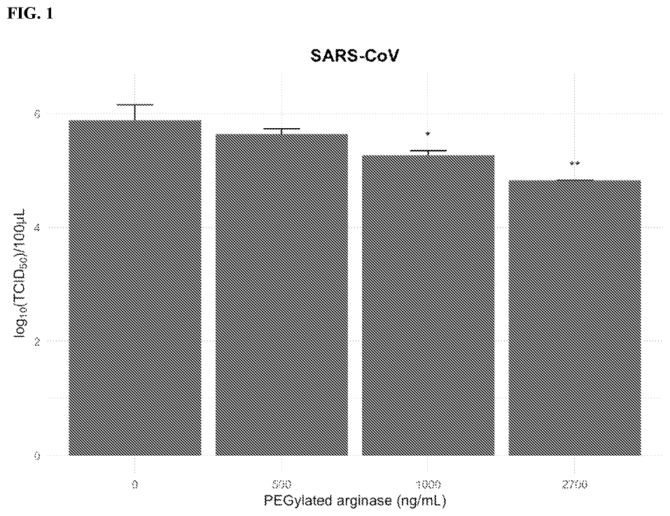

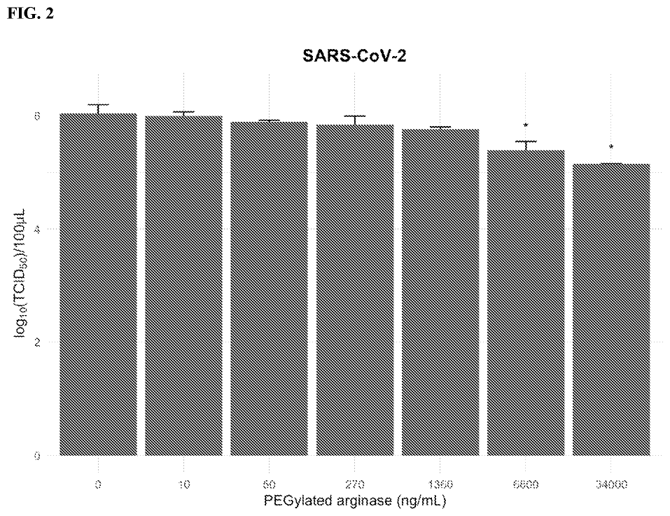

Dose-dependent inhibitory effect of PEGylated arginase on the replication of SAR-CoV and SARS-CoV-2 was observed (one-way ANOVA tests, p<0.01). Statistical significant inhibition of SARS-CoV and SARS-CoV-2 replication was observed with treatment of ≥1 μg/mL (FIG. 1) and ≥6.8 μg/mL (FIG. 2), respectively, of PEGylated arginase (Dunnett's tests, p<0.05).

### Example 2: Arginase Inhibits Replication of Middle East Respiratory Syndrome—Related Coronavirus (MERS-CoV)

In this example, the effect of recombinant arginase on replication of MERS-CoV is determined.

The non-PEGylated arginine used in this example was wild-type human arginase 1 with N-terminal 6xHis-tag was used (i.e., SEQ ID NO: 56). The PEGylated arginase used in this example was BCT-100, having the sequence of SEQ ID NO: 56 and made as described in U.S. Pat. No. 9,109,218, except that in this example wild-type human arginase 1 with N-terminal 6xHis-tag was used (i.e., SEQ ID NO: 56).

Huh-7 cells were maintained as a monolayer in Dulbecco's Modified Eagle Medium (DMEM) supplemented with 10% fetal bovine serum at 37° C. and 5% CO₂. The cells were resuspended in fresh complete growth medium at a density of 2×10⁵ cells/mL and seeded into a 24-well culture plate. Seeded cells were treated with 1250, 2500, 5000 or 10000 ng/mL of non-PEGylated or PEGylated arginase, and infected with MERS-CoV at MOI of 0.001. Culture supernatants were collected 24 hours-post-infection. Viral titre, in terms of the number of copy of viral genome, in the collected supernatants was quantified using quantitative reverse transcription PCR (RT-qPCR).

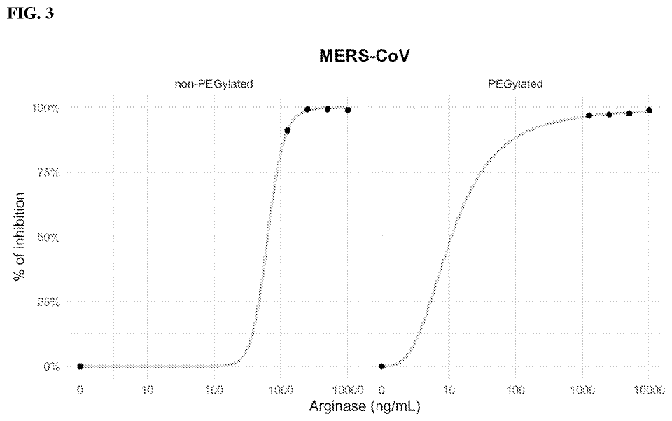

Inhibition of MERS-CoV replication was observed with all tested concentrations of arginase regardless of pegylation (FIG. 3). The half maximal inhibitory concentration (IC₅₀) was estimated to be ≤1325 ng/mL and ≤331 ng/mL (95% upper confidence limits) of non-PEGylated arginase and PEGylated arginase, respectively, via two-parameters logistic models.

### Example 3: Arginase Inhibits Replication of Influenza Virus

In this example, the effect of recombinant arginase on replication of influenza virus strains, including swine flu virus (H1N1) and avian flu virus (H5N1) is determined.

The non-PEGylated arginine used in this example was wild-type human arginase 1 with N-terminal 6xHis-tag was used (i.e., SEQ ID NO: 56). The PEGylated arginase used in this example was BCT-100, having the sequence of SEQ ID NO: 56 and made as described in U.S. Pat. No. 9,109,218, except that in this example wild-type human arginase 1 with N-terminal 6xHis-tag was used (i.e., SEQ ID NO: 56).

MDCK cells (ATCC) were cultured as monolayer in MEM supplemented with 10% fetal bovine serum at 37° C., 5% CO₂. Cells were resuspended in fresh medium at a density of 1×10⁵ cells/mL and seeded into a 24-well culture plate. Seeded cells were infected with influenza A virus, H1N1 (pandemic H1N1/09) or H5N1 (A/Viet Nam/1203/2004), at MOI of 0.01 and were treated with varying doses of no-PEGylated (for H1N1) or PEGylated (for H1N1 and H5N1) arginase. Culture supernatants were collected 24 hour post-infection. Titre of H1N1 and H5N1 in the collected supernatants was quantified using RT-qPCR and TCID₅₀ assay, respectively.

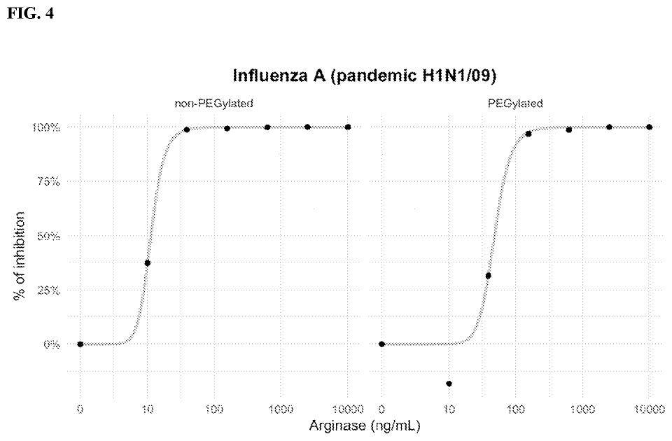

Replication of H1N1 was nearly completely abolished in the presence of ≥156 ng/mL of non-PEGylated or ≥625 ng/mL of PEGylated arginase (FIG. 4). ICH₅₀ of non-PEGylated or PEGylated arginase on H1N1 replication was estimated to be 11 ng/mL (96% confidence interval: 11-12 ng/mL) and 44 ng/mL (95% confidence interval: 11-181 ng/mL), respectively, via two-parameters logistic models.

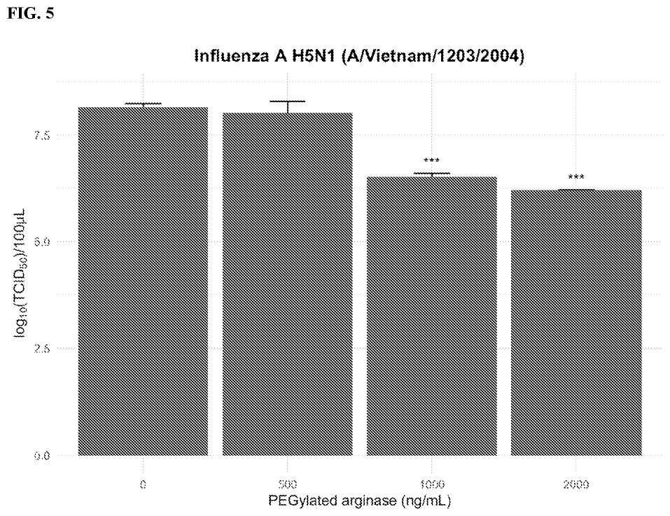

A similar dose-dependent inhibitory effect of PEGylated arginase on H5N1 replication was also observed (one-way ANOVA, F_{3,4}=91.24, p<0.001; FIG. 5). A 42-fold (i.e. 1.63−log₁₀) or 86-fold (i.e. 1.94−log₁₀) reduction in infectious titre was observed when treated with 1 μg/mL or 2 μg/mL, respectively, of PEGylated arginase (Dunnett's tests, p<0.001).

### Example 4: Arginase Inhibition of Human Adenovirus Replication

In this example, the effect of arginase on replication of adenovirus strains, including human adenovirus serotype 5 (HAdV-5) is determined.

The non-PEGylated arginine used in this example was wild-type human arginase 1 with N-terminal 6xHis-tag was used (i.e., SEQ ID NO: 56). The PEGylated arginase used in this example was BCT-100, having the sequence of SEQ ID NO: 56 and made as described in U.S. Pat. No. 9,109,218, except that in this example wild-type human arginase 1 with N-terminal 6xHis-tag was used (i.e., SEQ ID NO: 56).

HEp-2 cells (ATCC) are cultured as monolayer in MEM supplemented with 10% fetal bovine serum at 37° C., 5% CO₂. Cells are resuspended in fresh medium at a density of 1×10⁵ cells/mL and seeded into a 24-well culture plate. Seeded cells were treated with 10, 625, 1250, 2500, 5000 or 10000 ng/mL of non-PEGylated or PEGylated arginase and infected with HAdV-5 at a MOI of 0.5. Culture supernatants were collected 24 hour post-infection. Viral load in the collected supernatants was quantified in terms of the number of copy of viral genome using RT-qPCR.

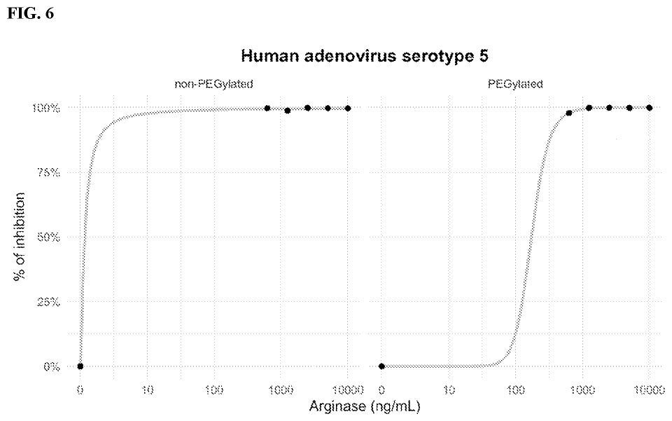

Replication of HAdV-5 was abolished in the presence of ≥625 ng/mL of non-PEGylated or 1250 ng/mL of PEGylated arginase (FIG. 6). ICH₅₀ of PEGylated arginase on HAdV-5 replication was estimated to be 139 ng/mL (95% confidence interval=40-477 ng/mL) via a two-parameters logistic model.

### INCORPORATION BY REFERENCE

Unless stated to the contrary, the entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of treating a virus-associated disease or disorder, the method comprising administering to a patient in need thereof a therapeutically effective amount of a composition comprising an arginase, or a pharmaceutically acceptable salt thereof,
wherein the arginase has at least about 90% sequence identity to SEQ ID NO:1 or 2, or a fragment thereof, and
wherein the virus-associated disease or disorder is associated with a virus selected from the group consisting of a coronavirus, a papillomavirus, a pneumovirus, a picornavirus, a flavivirus, an alphavirus, an ebolavirus, a morbillivirus, an enterovirus, an orthopneumovirus, a lentivirus, and a hepatovirus.

2. The method of claim 1, wherein the arginase is a PEGylated arginase, comprising at least one polyethylene glycol molecule conjugated to the arginase.

3. The method of claim 1 or claim 2, wherein the virus-associated disease or disorder is a virus infection selected from the group consisting of: a coronavirus infection, a papillomavirus infection, a pneumovirus infection, a picornavirus infection, a flavivirus infection, an alphavirus infection, an ebolavirus infection, a morbillivirus infection, an enterovirus infection, an orthopneumovirus infection, a lentivirus infection, and a hepatovirus infection.

4. A method of treating a virus-associated disease or disorder, the method comprising administering to a patient in need thereof a therapeutically effective amount of a composition comprising an arginase, or a pharmaceutically acceptable salt thereof,
wherein the arginase has at least about 90% sequence identity to a protein sequence selected from the group consisting of SEQ ID NO:3-50 and 56, or a fragment thereof.

5. The method of claim 4, wherein the arginase is a PEGylated arginase, comprising at least one polyethylene glycol molecule conjugated to the arginase.

6. The method of claim 4 or claim 5, wherein the virus-associated disease or disorder is associated with a virus selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebolavirus, a morbillivirus, an enterovirus, an orthopneumovirus, a lentivirus, and a hepatovirus.

7. The method of any one of claims 4-6, wherein the virus-associated disease or disorder is a virus infection selected from the group consisting of: an RNA virus infection, a DNA virus infection, a coronavirus infection, a papillomavirus infection, a pneumovirus infection, a picornavirus infection, an influenza virus infection, an adenovirus infection, a cytomegalovirus infection, a polyomavirus infection, a poxvirus infection, a flavivirus infection, an alphavirus infection, an ebolavirus infection, a morbillivirus infection, an enterovirus infection, an orthopneumovirus infection, a lentivirus infection, and a hepatovirus infection.

8. The method of any one of claims 1 to 7, wherein the virus-associated disease or disorder comprises a virus infection of an organ or a tissue of the patient.

9. The method of claim 8, wherein the organ or the tissue is selected from the group consisting of an eye, an ear, inner ear, a lung, trachea, bronchus, bronchioli, liver, gall bladder, bile duct, a kidney, bladder, a testicle, cervix, ovary, uterus, skin, and brain.

10. The method of any one of claims 1 to 9, wherein the virus-associated disease or disorder is selected from the group consisting of: acute respiratory distress syndrome; chronic obstructive pulmonary disease (COPD); pneumonia; drug-resistant pneumonia; hand, foot and mouth disease; atopic asthma; and non-atopic asthma.

11. A method of inhibiting genomic replication of a virus, the method comprising administering to a patient in need thereof a therapeutically effective amount of a composition comprising an arginase, or a pharmaceutically acceptable salt thereof,
wherein the arginase has at least about 90% sequence identity to SEQ ID NO:1 or 2, or a fragment thereof, and
wherein the virus is selected from the group consisting of a coronavirus, a papillomavirus, a pneumovirus, a picornavirus, a flavivirus, an alphavirus, an ebolavirus, a morbillivirus, an enterovirus, an orthopneumovirus, a lentivirus, and a hepatovirus.

12. The method of claim 11, wherein the arginase is a PEGylated arginase, comprising at least one polyethylene glycol molecule conjugated to the arginase.

13. A method of inhibiting genomic replication of a virus, the method comprising administering to a patient in need thereof a therapeutically effective amount of a composition comprising an arginase, or a pharmaceutically acceptable salt thereof,
wherein the arginase has at least about 90% sequence identity to a protein sequence selected from the group consisting of SEQ ID NO:3-50 and 56, or a fragment thereof.

14. The method of claim 13, wherein the arginase is a PEGylated arginase, comprising at least one polyethylene glycol molecule conjugated to the arginase.

15. The method of claim 13 or 14, wherein the virus is selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebolavirus, a morbillivirus, an enterovirus, an orthopneumovirus, a lentivirus, and a hepatovirus.

16. A method of inhibiting transmission of a virus, the method comprising administering to a patient in need thereof a therapeutically effective amount of a composition comprising an arginase,
wherein the arginase has at least about 90% sequence identity to a protein sequence selected from the group consisting of SEQ ID NO:1 or 2, or a pharmaceutically acceptable salt thereof, wherein the virus is selected from the group consisting of a coronavirus, a papillomavirus, a pneumovirus, a picornavirus, a flavivirus, an alphavirus, an ebolavirus, a morbillivirus, an enterovirus, an orthopneumovirus, a lentivirus, and a hepatovirus.

17. The method of claim 16, wherein the arginase is a PEGylated arginase, comprising at least one polyethylene glycol molecule conjugated to the arginase.

18. A method of inhibiting transmission of a virus, the method comprising administering to a patient in need thereof a therapeutically effective amount of a composition comprising an arginase, or a pharmaceutically acceptable salt thereof,
wherein the arginase has at least about 90% sequence identity to a protein sequence selected from the group consisting of SEQ ID NO:3-50 and 56, or a fragment thereof.

19. The method of claim 18, wherein the arginase is a PEGylated arginase, comprising at least one polyethylene glycol molecule conjugated to the arginase.

20. The method of claim 18 or 19, wherein the virus is selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebolavirus, a morbillivirus, an enterovirus, an orthopneumovirus, a lentivirus, and a hepatovirus.

21. A method of inhibiting assembly of a virus, the method comprising administering to a patient in need thereof a therapeutically effective amount of a composition comprising an arginase, or a pharmaceutically acceptable salt thereof,
wherein the arginase has at least about 90% sequence identity to a protein sequence selected from the group consisting of SEQ ID NO:1-50 and 56, or a fragment thereof.

22. The method of claim 21, wherein the arginase is a PEGylated arginase, comprising at least one polyethylene glycol molecule conjugated to the arginase.

23. A method of inhibiting virus gene expression, the method comprising administering to a patient in need thereof a therapeutically effective amount of a composition comprising an arginase, or a pharmaceutically acceptable salt thereof,
wherein the arginase has at least about 90% sequence identity to a protein sequence selected from the group consisting of SEQ ID NO:1-50 and 56, or a fragment thereof.

24. The method of claim 23, wherein the arginase is a PEGylated arginase, comprising at least one polyethylene glycol molecule conjugated to the arginase.

25. The method of claim 23 or 24, wherein the inhibiting comprises inhibiting gene expression, γ gene expression, or β gene expression and γ gene expression.

26. A method of inhibiting virus release, the method comprising administering to a patient in need thereof a therapeutically effective amount of a composition comprising an arginase, or a pharmaceutically acceptable salt thereof,
wherein the arginase has at least about 90% sequence identity to a protein sequence selected from the group consisting of SEQ ID NO:1-50 and 56, or a fragment thereof.

27. The method of claim 26, wherein the arginase is a PEGylated arginase, comprising at least one polyethylene glycol molecule conjugated to the arginase.

28. The method of any one of claims 23-27, wherein the virus is selected from the group consisting of an RNA virus, a DNA virus, a coronavirus, a papillomavirus, a pneumovirus, a picornavirus, an influenza virus, an adenovirus, a cytomegalovirus, a polyomavirus, a poxvirus, a flavivirus, an alphavirus, an ebolavirus, a morbillivirus, an enterovirus, an orthopneumovirus, a lentivirus, and a hepatovirus.

29. The method of any preceding claim, wherein the virus is a coronavirus.

30. The method of claim 29, wherein the coronavirus is selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2 (COVID-19).

31. The method of any one of claims 6-10, 13-15, and 18-28, wherein the virus is an influenza virus.

32. The method of claim 31, wherein the influenza virus is selected from the group consisting of influenza virus A (e.g., H1N1 and H5N1), influenza virus B, influenza virus C, influenza virus D.

33. The method of any one of claims 6-10, 13-15, and 18-28, wherein the virus is an adenovirus virus.

34. The method of claim 33, wherein the adenovirus virus is AdV5.

35. The method of any preceding claim, wherein the virus is a drug-resistant virus.

36. The method of any one of claims 1 to 35, further comprising administering a composition comprising an antiviral agent.

37. The method of claim 36, wherein the antiviral agent is selected from the group consisting of lamivudine, an interferon alpha composition, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), Rifampicin, zanamivir, peramivir, danoprevir, ritonavir, remdesivir, and oseltamivir.

38. The method of claim 36 or 37, wherein the administering of the antiviral agent is before, during, or after the administering of the arginase.

39. A method of treating a bacterial disease or disorder, the method comprising administering to a patient in need thereof a therapeutically effective amount of a composition comprising an arginase, or a pharmaceutically acceptable salt thereof,
wherein the arginase has at least about 90% sequence identity to SEQ ID NO:1 or 2, or a fragment thereof, and
wherein the bacterial disease or disorder is associated with a bacteria selected from the group consisting of: Streptococcus pneumoniae, Mycoplasma pneumoniae, Haemophilus influenzae, Legionella pneumophila, Salmonella enterica, Salmonella bongori, Escherichia coli, Helicobacter pylori, Neisseria gonorrhoeae, Neisseria meningitidis, Staphylococcus aureus, Acinetobacter baumannii, Burkholderia cepacian, Clostridium difficile, Clostridium sordellii, an Enterobacteriaceae, Enterococcus faecalis, Klebsiella pneumoniae, Morganella morganii, Mycobacterium abscessus, Mycobacterium tuberculosis, a Norovirus, Psuedomonas aeruginosa, and Stenotrophomonas maltophilia.

40. The method of claim 39, wherein the arginase is a PEGylated arginase, comprising at least one polyethylene glycol molecule conjugated to the arginase.

41. A method of treating a bacterial disease or disorder, the method comprising administering to a patient in need thereof a therapeutically effective amount of a composition comprising an arginase, or a pharmaceutically acceptable salt thereof,
wherein the arginase has at least about 90% sequence identity to a protein sequence selected from the group consisting of SEQ ID NO:3-50 and 56, or a fragment thereof.

42. The method of claim 41, wherein the arginase is a PEGylated arginase, comprising at least one polyethylene glycol molecule conjugated to the arginase.

43. The method of claim 41 or 42, wherein the bacterial disease or disorder is associated with a bacteria selected from the group consisting of: Chlamydia pneumoniae, Vibrio cholerae, Streptococcus pneumoniae, Mycoplasma pneumoniae, Haemophilus influenzae, Legionella pneumophila, Salmonella enterica, Salmonella bongori, Escherichia coli, Helicobacter pylori, Neisseria gonorrhoeae, Neisseria meningitidis, Staphylococcus aureus, Acinetobacter baumannii, Burkholderia cepacian, Clostridium difficile, Clostridium sordellii, an Enterobacteriaceae, Enterococcus faecalis, Klebsiella pneumoniae, Morganella morganii, Mycobacterium abscessus, Mycobacterium tuberculosis, a Norovirus, Psuedomonas aeruginosa, and Stenotrophomonas maltophilia.

44. A method of treating a fungal disease or disorder, the method comprising administering to a patient in need thereof a therapeutically effective amount of a composition comprising an arginase, or a pharmaceutically acceptable salt thereof,
wherein the arginase has at least about 90% sequence identity to a protein sequence selected from the group consisting of SEQ ID NO:1-50 and 56, or a fragment thereof.

45. The method of claim 44, wherein the arginase is a PEGylated arginase, comprising at least one polyethylene glycol molecule conjugated to the arginase.

46. The method of claim 44 or 45, wherein the fungal disease is associated with a fungus selected from the group consisting of a Pneumocystis fungus, an Aspergillus fungus, and a Candida fungus.

47. A method of treating an amoeba disease or disorder, the method comprising administering to a patient in need thereof a therapeutically effective amount of a composition comprising an arginase, or a pharmaceutically acceptable salt thereof,
wherein the arginase has at least about 90% sequence identity to a protein sequence selected from the group consisting of SEQ ID NO:1-50 and 56, or a fragment thereof.

48. The method of claim 47, wherein the arginase is a PEGylated arginase, comprising at least one polyethylene glycol molecule conjugated to the arginase.

49. The method of claim 47 or 48, wherein the amoeba disease or disorder is associated with an amoeba selected from the group consisting of Dientamoeba fragilis, Entamoeba histolytica, Naegleria fowleri, an Acanthamoeba, Acanthamoeba keratitis, Balamuthia mandrillaris, and Sappinia diploidea.

50. The method of any one of claims 1 to 49, wherein the arginase is administered at a dose of about 1 ng/kg body weight per day to about 1 mg/kg body weight per day.

51. The method of any one of claims 1 to 50, wherein the administering is topical, parenteral, oral, pulmonary, intratracheal, intranasal, intrathecal, transdermal, subcutaneous, intraocular, intravitreal, intraperitoneal, intraduodenal, or by inhalation.

52. The method of any one of claims 1 to 51, wherein the arginase has at least about 90% sequence identity to a protein sequence selected from the group consisting of SEQ ID NO:3-43, or a fragment thereof, and wherein the arginase includes a protein tag sequence.

53. The method of claim 52, wherein the protein tag sequence comprises a 6×His tag sequence of SEQ ID NO:51.

54. The method of claim 52 or 53, wherein the protein tag sequence is located at the amino terminus of the arginase.

55. The method of claim 52 or 53, wherein the protein tag sequence is located at the carboxy terminus of the arginase.

56. The method of any one of claims 1 to 55, wherein the arginase is a PEGylated-arginase comprising 2, 3, 4, or more polyethylene glycol molecules conjugated to the arginase.

57. The method of any one of claims 2, 3, 5-10, 12, 14, 15, 17, 19, 20, 22, 24, 25, 27-38, 40, 42, 43, 45, 46, and 48-56, wherein the polyethylene glycol molecule is about 5 kDa, about 10 kDa, about 15 kDa, about 20 kDa, about 30 kDa, or about 40 kDa.

58. The method of any one of claims 2, 3, 5-10, 12, 14, 15, 17, 19, 20, 22, 24, 25, 27-38, 40, 42, 43, 45, 46, and 48-56, wherein the polyethylene glycol is from about 10 kDa to about 30 kDa or from about 20 kDa to about 40 kDa.

59. The method of any one of claims 1 to 58, wherein the composition further comprises a non-native metal cofactor.

60. The method of claim 59, wherein the non-native metal cofactor is selected from the group consisting of cobalt, manganese, iron, and zinc.

61. A composition comprising:
an arginase, or a pharmaceutically acceptable salt thereof,
wherein the arginase has at least about 90% sequence identity to a protein sequence selected from the group consisting of SEQ ID NO:1-50 and 56, or a fragment thereof;
an antiviral agent; and
a pharmaceutically acceptable excipient.

62. The composition of claim 61, wherein the arginase is a PEGylated arginase, comprising at least one polyethylene glycol molecule conjugated to the arginase.

63. The composition of claim 61 or 62, wherein the antiviral agent is selected from the group consisting of lamivudine, an interferon alpha composition, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), Rifampicin, zanamivir, peramivir, danoprevir, ritonavir, remdesivir, and oseltamivir.

64. The composition of any one of claims 61-63, wherein the arginase has at least about 90% sequence identity to a protein sequence selected from the group consisting of SEQ ID NO:3-43, or a fragment thereof, and wherein the arginase includes a protein tag sequence.

65. The composition of claim 64, wherein the protein tag sequence comprises a 6×His tag sequence of SEQ ID NO:51.

66. The composition of claim 64 or 65, wherein the protein tag sequence is located at the amino terminus of the arginase.

67. The composition of claim 64 or 65, wherein the protein tag sequence is located at the carboxy terminus of the arginase.

68. The composition of any one of claims 62 to 67, wherein the PEGylated arginase comprises 2, 3, 4, or more polyethylene glycol molecules conjugated to the arginase sequence.

69. The composition of any one of claims 62-68, wherein the polyethylene glycol molecule is about 5 kDa, about 10 kDa, about 15 kDa, about 20 kDa, about 30 kDa, or about 40 kDa.

70. The composition of any one of claims 62-68, wherein the polyethylene glycol molecule is from about 10 kDa to about 30 kDa or from about 20 kDa to about 40 kDa.

71. The composition of any one of claims 61 to 70, wherein the composition further comprises a non-native metal cofactor.

72. The composition of claim 71, wherein the non-native metal cofactor is selected from the group consisting of cobalt, manganese, iron, and zinc.

73. A kit comprising:
an arginase, or a pharmaceutically acceptable salt thereof,
wherein the arginase has at least about 90% sequence identity to a protein sequence selected from the group consisting of SEQ ID NO:1-50 and 56, or a fragment thereof; and
buffers, reagents, and detailed instructions for inhibiting production of a virus.

74. The kit of claim 73, wherein the arginase is a PEGylated arginase, comprising at least one polyethylene glycol molecule conjugated to the arginase.

75. The kit of claim 73 or 74, further comprising an antiviral agent.

76. The kit of claim 75, wherein the antiviral agent is selected from the group consisting of lamivudine, an interferon alpha composition, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), Rifampicin, zanamivir, peramivir, danoprevir, ritonavir, remdesivir, and oseltamivir.

77. The kit of any one of claims 73 to 76, wherein the kit is for inhibiting production of a coronavirus.

78. The kit of claim 77, wherein the coronavirus is selected from the group consisting of:
229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2 (COVID-19).

79. The kit of any one of claims 73-76, wherein the virus is an influenza virus.

80. The kit of claim 79, wherein the influenza virus is selected from the group consisting of influenza virus A (e.g., H1N1 and H5N1), influenza virus B, influenza virus C, influenza virus D.

81. The kit of any one of claims 73-76, wherein the virus is an adenovirus virus.

82. The kit of claim 81, wherein the adenovirus virus is AdV5.

83. The kit of any one of claims 73-82, wherein the arginase has at least about 90% sequence identity to a protein sequence selected from the group consisting of SEQ ID NO:3-43, or a fragment thereof, and wherein the arginase includes a protein tag sequence.

84. The kit of claim 83, wherein the protein tag sequence is a 6×His tag sequence of SEQ ID NO:51.

85. The kit of claim 83 or 84, wherein the protein tag sequence is located at the amino terminus of the arginase.

86. The kit of claim 83 or 84, wherein the protein tag sequence is located at the carboxy terminus of the arginase.

87. The kit of any one of claims 74 to 86, wherein the PEGylated-arginase comprises 2, 3, 4, or more polyethylene glycol molecules conjugated to the arginase sequence.

88. The kit of any one of claims 74 to 87, wherein the polyethylene glycol is about 5 kDa, about 10 kDa, about 15 kDa, about 20 kDa, about 30 kDa, or about 40 kDa.

89. The kit of any one of claims 74 to 87, wherein the polyethylene glycol is from about 10 kDa to about 30 kDa or from about 20 kDa to about 40 kDa.

90. The kit of any one of claims 73 to 89, wherein the kit further comprises a non-native metal cofactor.

91. The kit of claim 90, wherein the non-native metal cofactor is selected from the group consisting of cobalt, manganese, iron, and zinc.


SEQUENCE LISTING (57 sequences)

SEQ ID NO: 1 - protein, 322 aa, Artificial Sequence
  Description of Artificial Sequence Synthetic polypeptide
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 2 - protein, 134 aa, Artificial Sequence
  Description of Artificial Sequence Synthetic polypeptide
HSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVD

SEQ ID NO: 3 - protein, 322 aa, Artificial Sequence
  Description of Artificial Sequence Synthetic polypeptide
MSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 4 - protein, 322 aa, Artificial Sequence
  Description of Artificial Sequence Synthetic polypeptide
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 5 - protein, 322 aa, Artificial Sequence
  Description of Artificial Sequence Synthetic polypeptide
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 6 - protein, 322 aa, Artificial Sequence
  Description of Artificial Sequence Synthetic polypeptide
MSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 7 - protein, 322 aa, Artificial Sequence
  Description of Artificial Sequence Synthetic polypeptide
MSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 8 - protein, 322 aa, Artificial Sequence
  Description of Artificial Sequence Synthetic polypeptide
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 9 - protein, 322 aa, Artificial Sequence
  Description of Artificial Sequence Synthetic polypeptide
MSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 10 - protein, 298 aa, Artificial Sequence
  Description of Artificial Sequence Synthetic polypeptide
MKPISIIGVPMDLGQTRRGVDMGPSAMRYAGVIERLERLHYDIEDLGDIPIGKAERLHEQGDSRLRNLKAVAEANEKLAAAVDQWQRGRFPLVLGGDHSIAIGTLAGVAKHYERLGVIWYDAHGDVNTAETSPSGNIHGMPLAASLGFGHPALTQIGGYSPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLM

SEQ ID NO: 11 - protein, 304 aa, Artificial Sequence
  Description of Artificial Sequence Synthetic polypeptide
MKPISIIGVPMDLGQTRRGVDMGPSAMRYAGVIERLERLHYDIEDLGDIPIGKAERLHEQGDSRLRNLKAVAEANEKLAAAVDQWQRGRFPLVLGGDHSIAIGTLAGVAKHYERLGVIWYDAHGDVNTAETSPSGNIHGMPLAASLGFGHPALTQIGGYCPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLMHHHHHH

SEQ ID NO: 12 - protein, 322 aa, Artificial Sequence
  Description of Artificial Sequence Synthetic polypeptide
MSAKSRTIGIIGAPFSKGQPRGGVEEGPTVLRKAGLLEKLKEQESDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 13 - protein, 322 aa, Artificial Sequence
  Description of Artificial Sequence Synthetic polypeptide
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 14 - protein, 322 aa, Artificial Sequence
  Description of Artificial Sequence Synthetic polypeptide
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 15 - protein, 322 aa, Artificial Sequence
  Description of Artificial Sequence Synthetic polypeptide
MSAKSRTIGIIGAPFSKGQPRGGVEEGPTVLRKAGLLEKLKEQESDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 16 - protein, 322 aa, Artificial Sequence
  Description of Artificial Sequence Synthetic polypeptide
MSAKSRTIGIIGAPFSKGQPRGGVEEGPTVLRKAGLLEKLKEQESDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 17 - protein, 322 aa, Artificial Sequence
  Description of Artificial Sequence Synthetic polypeptide
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 18 - protein, 322 aa, Artificial Sequence
  Description of Artificial Sequence Synthetic polypeptide
MSAKSRTIGIIGAPFSKGQPRGGVEEGPTVLRKAGLLEKLKEQESDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 19 - protein, 322 aa, Artificial Sequence
  Description of Artificial Sequence Synthetic polypeptide
MSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 20 - protein, 322 aa, Artificial Sequence
  Description of Artificial Sequence Synthetic polypeptide
MSAKSRTIGIIGAPFSKGQPRGGVEEGPTVLRKAGLLEKLKEQESDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 21 - protein, 322 aa, Artificial Sequence
  Description of Artificial Sequence Synthetic polypeptide
MSAKSRTIGIIGAPFSKGQPRGGVEEGPTVLRKAGLLEKLKEQESDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 22 - protein, 322 aa, Artificial Sequence
  Description of Artificial Sequence Synthetic polypeptide
MSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 23 - protein, 322 aa, Artificial Sequence
  Description of Artificial Sequence Synthetic polypeptide
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 24 - protein, 322 aa, Artificial Sequence
  Description of Artificial Sequence Synthetic polypeptide
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 25 - protein, 322 aa, Artificial Sequence
  Description of Artificial Sequence Synthetic polypeptide
MSAKSRTIGIIGAPFSKGQPRGGVEEGPTVLRKAGLLEKLKEQESDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 26 - protein, 322 aa, Artificial Sequence
  Description of Artificial Sequence Synthetic polypeptide
MSAKSRTIGIIGAPFSKGQPRGGVEEGPTVLRKAGLLEKLKEQESDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 27 - protein, 322 aa, Artificial Sequence
  Description of Artificial Sequence Synthetic polypeptide
MSAKSRTIGIIGAPFSKGQPRGGVEEGPTVLRKAGLLEKLKEQESDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 28 - protein, 322 aa, Artificial Sequence
  Description of Artificial Sequence Synthetic polypeptide
MSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 29 - protein, 322 aa, Artificial Sequence
  Description of Artificial Sequence Synthetic polypeptide
MSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 30 - protein, 322 aa, Artificial Sequence
  Description of Artificial Sequence Synthetic polypeptide
MSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 31 - protein, 322 aa, Artificial Sequence
  Description of Artificial Sequence Synthetic polypeptide
MSFKSQSIGIIGAPFSKGQPRGGVEEGPTALRKAGLLEKLKEQECDVKDYGDLCFADVPNDTPFQIVKNPRSVGKANQQLADVVAEIKKNGRTSLVLGGDHSMAIGSISGHARVHPDLCVIWVDAHTDINTPLTTTTGNLHGQPVSFLLKELKEKIPEVPGLSWVTPCLSAKDIVYIGLRDVDPAEHYILKTLGIKYFSMIEVDKLGIGKVMEEAFSYLLGRKKRPIHLSFDVDGLDPFFTPATGTPVHGGLSYREGIYITEEIYKTGLLSGLDIMEVNPSLGKTPEEVTRTVNTAVALVLACFGVAREGNHKPIDYLKPPK

SEQ ID NO: 32 - protein, 322 aa, Artificial Sequence
  Description of Artificial Sequence Synthetic polypeptide
MSFKSQSIGIIGAPFSKGQPRGGVEEGPTALRKAGLLEKLKEQECDVKDYGDLSFADVPNDTPFQIVKNPRSVGKANQQLADVVAEIKKNGRTSLVLGGDHSMAIGSISGHARVHPDLSVIWVDAHTDINTPLTTTTGNLHGQPVSFLLKELKEKIPEVPGLSWVTPSLSAKDIVYIGLRDVDPAEHYILKTLGIKYFSMIEVDKLGIGKVMEEAFSYLLGRKKRPIHLSFDVDGLDPFFTPATGTPVHGGLSYREGIYITEEIYKTGLLSGLDIMEVNPSLGKTPEEVTRTVNTAVALVLASFGVAREGNHKPIDYLKPPK

SEQ ID NO: 33 - protein, 321 aa, Artificial Sequence
  Description of Artificial Sequence Synthetic polypeptide
SAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 34 - protein, 321 aa, Artificial Sequence
  Description of Artificial Sequence Synthetic polypeptide
  MOD_RES (1)..(1): Met or absent
XSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPP

SEQ ID NO: 35 - protein, 333 aa, Artificial Sequence
  Description of Artificial Sequence Synthetic polypeptide
SVHSVAVIGAPFSQGQKRKGVEHGPAAIREAGLMKRLSSLGCHLKDFGDLSFTPVPKDDLYNNLIVNPRSVGLANQELAEVVSRAVSDGYSCVTLGGDHSLAIGTISGHARHCPDLCVVWVDAHADINTPLTTSSGNLHGQPVSFLLRELQDKVPQLPGFSWIKPCISSASIVYIGLRDVDPPEHFILKNYDIQYFSMRDIDRLGIQKVMERTFDLLIGKRQRPIHLSFDIDAFDPTLAPATGTPVVGGLTYREGMYIAEEIHNTGLLSALDLVEVNPQLATSEEEAKTTANLAVDVIASSFGQTREGGHIVYDQLPTPSSPDESENQARVRI

SEQ ID NO: 36 - protein, 321 aa, Artificial Sequence
  Description of Artificial Sequence Synthetic polypeptide
  MOD_RES (1)..(1): Met or absent
XSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRSVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPP

SEQ ID NO: 37 - protein, 321 aa, Artificial Sequence
  Description of Artificial Sequence Synthetic polypeptide
  MOD_RES (1)..(1): Met or absent
XSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLCFDVDGLDPSFTPATGTPVVGGLTYREGLYITEEIYKTGLLSGLDIMEVNPSLGKTPEEVTRTVNTAVAITLACFGLAREGNHKPIDYLNPP

SEQ ID NO: 38 - protein, 321 aa, Artificial Sequence
  Description of Artificial Sequence Synthetic polypeptide
  MOD_RES (1)..(1): Met or absent
XSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLGFDVDGLDPSFTPATGTPVVGGLTYREGLYITEEIYKTGLLSGLDIMEVNPSLGKTPEEVTRTVNTAVAITLACFGLAREGNHKPIDYLNPP

SEQ ID NO: 39 - protein, 321 aa, Artificial Sequence
  Description of Artificial Sequence Synthetic polypeptide
  MOD_RES (1)..(1): Met or absent
XSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAFFGLAREGNHKPIDYLNPP

SEQ ID NO: 40 - protein, 321 aa, Artificial Sequence
  Description of Artificial Sequence Synthetic polypeptide
  MOD_RES (1)..(1): Met or absent
XSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAIFGLAREGNHKPIDYLNPP

SEQ ID NO: 41 - protein, 321 aa, Artificial Sequence
  Description of Artificial Sequence Synthetic polypeptide
  MOD_RES (1)..(1): Met or absent
XSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLREVDPGEHYILKTLGIKYFSMTEVDRLGIGKVMEETLSYLLGRKKRPIHLAFDVDGLDPSFTPATGTPVVGGLTYREGLYITEEIYKTGLLSGLDIMEVNPSLGKTPEEVTRTVNTAVAITLACFGLAREGNHKPIDYLNPP

SEQ ID NO: 42 - protein, 138 aa, Artificial Sequence
  Description of Artificial Sequence Synthetic polypeptide
FPLVLGGDHSIAIGTLAGVAKHYERLGVIWYDAHGDVNTAETSPSGNIHGMPLAASLGFGHPALTQIGGYSPKIKPEHVVLIGVRSLDEGEKKFIREKGIKIYTMHEVDRLGMTRVMEETIAYLKERTDGVHLSLDLD

SEQ ID NO: 43 - protein, 134 aa, Artificial Sequence
  Description of Artificial Sequence Synthetic polypeptide
HSMAIGSISGHARVHPDLCVIWVDAHTDINTPLTTTTGNLHGQPVSFLLKELKEKIPEVPGLSWVTPCLSAKDIVYIGLRDVDPAEHYILKTLGIKYFSMIEVDKLGIGKVMEEAFSYLLGRKKRPIHLSFDVD

SEQ ID NO: 44 - protein, 329 aa, Artificial Sequence
  Description of Artificial Sequence Synthetic polypeptide
MHHHHHHMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 45 - protein, 329 aa, Artificial Sequence
  Description of Artificial Sequence Synthetic polypeptide
MHHHHHHMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 46 - protein, 329 aa, Artificial Sequence
  Description of Artificial Sequence Synthetic polypeptide
MHHHHHHMSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 47 - protein, 329 aa, Artificial Sequence
  Description of Artificial Sequence Synthetic polypeptide
MHHHHHHMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 48 - protein, 329 aa, Artificial Sequence
  Description of Artificial Sequence Synthetic polypeptide
MHHHHHHMSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 49 - protein, 329 aa, Artificial Sequence
  Description of Artificial Sequence Synthetic polypeptide
MHHHHHHMSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 50 - protein, 329 aa, Artificial Sequence
  Description of Artificial Sequence Synthetic polypeptide
MHHHHHHMSAKSRTIGIIGAPFSKGQPRGGVEEGPTVLRKAGLLEKLKEQEADVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPAISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLAAFGLAREGNHKPIDYLNPPK

SEQ ID NO: 51 - protein, 6 aa, Artificial Sequence
  Description of Artificial Sequence Synthetic 6xHis tag
HHHHHH

SEQ ID NO: 52 - protein, 8 aa, Artificial Sequence
  Description of Artificial Sequence Synthetic peptide
DYKDDDDK

SEQ ID NO: 53 - protein, 14 aa, Artificial Sequence
  Description of Artificial Sequence Synthetic peptide
GKPIPNPLLGLDST

SEQ ID NO: 54 - protein, 10 aa, Artificial Sequence
  Description of Artificial Sequence Synthetic peptide
EQKLISEEDL

SEQ ID NO: 55 - protein, 9 aa, Artificial Sequence
  Description of Artificial Sequence Synthetic peptide
YPYDVPDYA

SEQ ID NO: 56 - protein, 329 aa, Artificial Sequence
  Description of Artificial Sequence Synthetic polypeptide
MHHHHHHMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 57 - protein, 7 aa, Artificial Sequence
  Description of Artificial Sequence Synthetic peptide
MHHHHHH